SEQUENCE LISTING (22 sequences)

SEQ ID NO: 1 - protein, 280 aa, Homo sapiens
  CD37
MSAQESCLSLIKYFLFVFNLFFFVLGSLIFCFGIWILIDKTSFVSFVGLAFVPLQIWSKVLAISGIFTMGIALLGCVGALKELRCLLGLYFGMLLLLFATQITLGILISTQRAQLERSLRDVVEKTIQKYGTNPEETAAEESWDYVQFQLRCCGWHYPQDWFQVLILRGNGSEAHRVPCSCYNLSATNDSTILDKVILPQLSRLGHLARSRHSADICAVPAESHIYREGCAQGLQKWLHNNLISIVGICLGVGLLELGFMTLSIFLCRNLDHVYNRLAYR

SEQ ID NO: 2 - protein, 281 aa, Macaca mulatta
  CD37
MSAQESCLSLIKYFLFVFNLFFFVILGSLIFCFGIWILIDKTSFVSFVGLAFVPLQIWSKVLAISGVFTMGLALLGCVGALKELRCLLGLYFGMLLLLFATQITLGILISTQRAQLERSLQDIVEKTIQRYHTNPEETAAEESWDYVQFQLRCCGWHSPQDWFQVLTLRGNGSEAHRVPCSCYNLSATNDSTILDKVILPQLSRLGQLARSRHSTDICAVPANSHIYREGCARSLQKWLHNNLISIVGICLGVGLLELGFMTLSIFLCRNLDHVYNRLRYR

SEQ ID NO: 3 - protein, 281 aa, Mus musculus
  CD37
MSAQESCLSLIKYFLFVFNLFFFVLGGLIFCFGTWILIDKTSFVSFVGLSFVPLQTWSKVLAVSGVLTMALALLGCVGALKELRCLLGLYFGMLLLLFATQITLGILISTQRVRLERRVQELVLRTIQSYRTNPDETAAEESWDYAQFQLRCCGWQSPRDWNKAQMLKANESEEPFVPCSCYNSTATNDSTVFDKLFFSQLSRLGPRAKLRQTADICALPAKAHIYREGCAQSLQKWLHNNIISIVGICLGVGLLELGFMTLSIFLCRNLDHVYDRLARYR

SEQ ID NO: 4 - protein, 5 aa, Artificial Sequence
  VH-CDR1, Antibody CD37-3
TSGVS

SEQ ID NO: 5 - protein, 9 aa, Artificial Sequence
  VH-CDR2, Antibody CD37-3
VIWGDGSTN

SEQ ID NO: 6 - protein, 7 aa, Artificial Sequence
  VH-CDR3, Antibody CD37-3
GGYSLAH

SEQ ID NO: 7 - protein, 11 aa, Artificial Sequence
  VL-CDR1, Antibody CD37-3
RASENIRSNLA

SEQ ID NO: 8 - protein, 7 aa, Artificial Sequence
  VL-CDR2, Antibody CD37-3
VATNLAD

SEQ ID NO: 9 - protein, 9 aa, Artificial Sequence
  VL-CDR3, Antibody CD37-3
QHYWGTTWT

SEQ ID NO: 10 - protein, 115 aa, Artificial Sequence
  VH amino acid sequence, Antibody muCD37-3
QVQVKESGPGLVAPSQSLSITCTVSGFSLTTSGVSWVRQPPGKGLEWLGVIWGDGSTNYHSALKSRLSIKKDHSKSQVFLKLNSLQTDDTATYYCAKGGYSLAHWGQGTLVTVSA

SEQ ID NO: 11 - protein, 115 aa, Artificial Sequence; identical to SEQ ID NO: 10
  VH amino acid sequence, Antibody chCD37-3

SEQ ID NO: 12 - protein, 115 aa, Artificial Sequence
  VH amino acid sequence, Antibody huCD37-3 (version 1.0)
QVQVQESGPGLVAPSQTLSITCTVSGFSLTTSGVSWVRQPPGKGLEWLGVIWGDGSTNYHPSLKSRLSIKKDHSKSQVFLKLNSLTAADTATYYCAKGGYSLAHWGQGTLVTVSS

SEQ ID NO: 13 - protein, 108 aa, Artificial Sequence
  VL amino acid sequence, Antibody muCD37-3
DIQMTQSPASLSVSVGETVTITCRASENIRSNLAWYQQKQGKSPQLLVNVATNLADGVPSRFSGSGSGTQYSLKINSLQSEDFGTYYCQHYWGTTWTFGGGTKLEIKR

SEQ ID NO: 14 - protein, 108 aa, Artificial Sequence; identical to SEQ ID NO: 13
  VL amino acid sequence, Antibody chCD37-3

SEQ ID NO: 15 - protein, 108 aa, Artificial Sequence
  VL amino acid sequence, Antibody huCD37-3
DIQMTQSPSSLSVSVGERVTITCRASENIRSNLAWYQQKPGKSPKLLVNVATNLADGVPSRFSGSGSGTDYSLKINSLQPEDFGTYYCQHYWGTTWTFGQGTKLEIKR

SEQ ID NO: 16 - protein, 445 aa, Artificial Sequence
  Full-length heavy chain amino acid sequence, Antibody muCD37-3
QVQVKESGPGLVAPSQSLSITCTVSGFSLTTSGVSWVRQPPGKGLEWLGVIWGDGSTNYHSALKSRLSIKKDHSKSQVFLKLNSLQTDDTATYYCAKGGYSLAHWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 17 - protein, 445 aa, Artificial Sequence
  Full-length heavy chain amino acid sequence, Antibody chCD37-3
QVQVKESGPGLVAPSQSLSITCTVSGFSLTTSGVSWVRQPPGKGLEWLGVIWGDGSTNYHSALKSRLSIKKDHSKSQVFLKLNSLQTDDTATYYCAKGGYSLAHWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 444 aa, Artificial Sequence
  Full-length heavy chain amino acid sequence, Antibody huCD37-3
QVQVQESGPGLVAPSQTLSITCTVSGFSLTTSGVSWVRQPPGKGLEWLGVIWGDGSTNYHPSLKSRLSIKKDHSKSQVFLKLNSLTAADTATYYCAKGGYSLAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 214 aa, Artificial Sequence
  Full-length light chain amino acid sequence, Antibody muCD37-3
DIQMTQSPASLSVSVGETVTITCRASENIRSNLAWYQQKQGKSPQLLVNVATNLADGVPSRFSGSGSGTQYSLKINSLQSEDFGTYYCQHYWGTTWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 20 - protein, 214 aa, Artificial Sequence
  Full-length light chain amino acid sequence, Antibody chCD37-3
DIQMTQSPASLSVSVGETVTITCRASENIRSNLAWYQQKQGKSPQLLVNVATNLADGVPSRFSGSGSGTQYSLKINSLQSEDFGTYYCQHYWGTTWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 214 aa, Artificial Sequence
  Full-length light chain amino acid sequence, Antibody huCD37-3
DIQMTQSPSSLSVSVGERVTITCRASENIRSNLAWYQQKPGKSPKLLVNVATNLADGVPSRFSGSGSGTDYSLKINSLQPEDFGTYYCQHYWGTTWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 115 aa, Artificial Sequence
  VH amino acid sequence, Antibody huCD37-3 (version 1.1)
QVQVQESGPGLVAPSQTLSITCTVSGFSLTTSGVSWVRQPPGKGLEWLGVIWGDGSTNYHSSLKSRLSIKKDHSKSQVFLKLNSLTAADTATYYCAKGGYSLAHWGQGTLVTVSS

(19) United States
(12) Patent Application Publication
(10) Pub. No.: US 2015/0343077 A1
(43) Pub. Date: Dec. 3, 2015

(54) Anti-CD37 Immunoconjugate Dosing Regimens

(71) Applicant: ImmunoGen, Inc., Waltham, MA (US)

(72) Inventors: Jutta DECKERT, Lexington, MA (US); Robert J. LUTZ, Wayland, MA (US); Joanne Elizabeth Sarah SCHINDLER, Natick, MA (US); Rodrigo Ricardo RUIZ SOTO, Boston, MA (US)

(73) Assignee: ImmunoGen, Inc.

(21) Appl. No.: 14/710,354

(22) Filed: May 12, 2015

Related U.S. Application Data
(60) Provisional application No. 62/088,237, filed on Dec. 5, 2014.
(60) Provisional application No. 62/075,780, filed on Nov. 5, 2014.
(60) Provisional application No. 62/004,754, filed on May 29, 2014.
(60) Provisional application No. 61/992,848, filed on May 13, 2014.

(51) Int. Cl. A61K 47/48; A61K 9/00; C07K 16/30; A61K 31/5365; A61K 31/56; A61K 38/19

(52) U.S. Cl. CPC A61K 47/4863; A61K 31/56; A61K 38/193; C07K 16/3061; A61K 31/5365; A61K 9/0019; C07K 2317/565; C07K 2317/56; A61K 2039/505

(57) ABSTRACT

Methods of administering immunoconjugates that bind to CD37 are provided. The methods comprise administering an anti-CD37 immunoconjugate to a person in need thereof, for example, a cancer patient, at a therapeutically effective dosing regimen that results in minimal adverse effects.

### CROSS REFERENCE TO RELATED APPLICATIONS

Related applications U.S. 61/992,848, filed May 13, 2014, U.S. 62/004,754, filed May 29, 2014, U.S. 62/075,780, filed Nov. 5, 2014, and U.S. 62/088,237, filed Dec. 5, 2014 are all incorporated herein by reference in their entireties.

### REFERENCE TO A SEQUENCE LISTING SUBMITTED ELECTRONICALLY VIA EFS-WEB

The content of the electronically submitted sequence listing (Name: 2921_{—}0590004_SeqListing_ST25, Size: 34,534 bytes; and Date of Creation: May 11, 2015), filed with the application is incorporated by reference in its entirety.

### FIELD OF THE INVENTION

The field of the invention generally relates to methods of administering anti-CD37 immunoconjugates for the treatment of diseases, such as cancer.

### BACKGROUND OF THE INVENTION

Cancer is one of the leading causes of death in the developed world, with over one million people diagnosed with cancer and 500,000 deaths per year in the United States alone. Overall it is estimated that more than 1 in 3 people will develop some form of cancer during their lifetime.

Leukocyte antigen CD37 (“CD37”), also known as GP52-40, tetraspanin-26, or TSPAN26, is expressed on B cells during the pre-B to peripheral mature B-cell stages, but is absent on terminal differentiation to plasma cells. (Link et al., 1987, J Pathol. 152:12-21). The CD37 antigen is only weakly expressed on T-cells, myeloid cells and granulocytes (Schwartz-Albiez et al. 1988, J. Immunol, 140(3)905-914). However, CD37 is also expressed on malignant B-cells such as those found in non-Hodgkin's lymphoma (NHL) and chronic lymphoid leukemia (CLL) (Moore et al. 1986, J Immunol 137(9):3013-8). This expression profile suggests that CD37 represents a promising therapeutic target for B-cell malignancies, and currently, there is a clear unmet medical need for more effective therapeutics for B-cell malignancies.

### BRIEF SUMMARY OF THE INVENTION

Methods of administering an anti-CD37 immunoconjugate are provided herein. Thus, described herein are methods for treating a patient having cancer, e.g., a B-cell malignancy, comprising administering to the patient an effective dose of an immunoconjugate which binds to CD37, wherein the immunoconjugate is administered at a dose of about 0.1 mg/kg to about 3.0 mg/kg body weight. In some embodiments, the anti-CD37 immunoconjugate comprises the antibody CD37-3, the linker SMCC, and the maytansinoid DM1 (IMGN529).

In some embodiments, the immunoconjugate is administered with a corticosteroid to prevent side effects. In some embodiments, the corticosteroid is administered to reduce cytokine-mediated adverse events including, for example, neutropenia and febrile neutropenia. Thus, in some embodiments, the corticosteroid is administered to decrease, shorten, or prevent neutropenia or febrile neutropenia. In some embodiments, the neutropenia presents early in the dosing cycle. In some embodiments, the neutropenia is febrile neutropenia.

In some embodiments, the corticosteroid is administered peri-infusionally. In some embodiments, the corticosteroid is administered prior to administration of the anti-CD37 immunoconjugate and on days 1-3 of the anti-CD37 immunoconjugate administration cycle. In some embodiments, the corticosteroid is administered 30 to 60 minutes prior to administration of the anti-CD37 immunoconjugate and on days 1-3 of the anti-CD37 immunoconjugate administration cycle. In some embodiments, the corticosteroid is administered intravenously 30 to 60 minutes prior to administration of the anti-CD37 immunoconjugate and orally on days 1-3 of the anti-CD37 immunoconjugate administration cycle. In some embodiments, the corticosteroid is administered prior to administration of the anti-CD37 immunoconjugate and on days 2 and 3 of the anti-CD37 immunoconjugate administration cycle. In some embodiments, the corticosteroid is administered 30 to 60 minutes prior to administration of the anti-CD37 immunoconjugate and on days 2 and 3 of the anti-CD37 immunoconjugate administration cycle. In some embodiments, the corticosteroid is administered intravenously 30 to 60 minutes prior to administration of the anti-CD37 immunoconjugate and orally on days 2 and 3 of the anti-CD37 immunoconjugate administration cycle. In some embodiments, the corticosteroid is dexamethasone. In some embodiments, 10 mg dexamethasone is administered intravenously and/or 8 mg dexamethasone is administered orally. In some embodiments, 10 mg dexamethasone is administered intravenously 30 to 60 minutes prior to the administration of the anti-CD37 immunoconjugate (e.g., IMGN529) and 8 mg dexamethasone is administered orally on days 2 and 3 of a 3-week anti-CD37 immunoconjugate administration cycle.

In some embodiments, the immunoconjugate is administered with a growth factor. In some embodiments, the growth factor is administered to prevent neutropenia. In some embodiments, the neutropenia presents late in the dosing cycle.

In some embodiments, the immunoconjugate is administered with a corticosteroid and a growth factor.

In some embodiments, the immunoconjugate is administered with at least one compound that reduces or inhibits the release, level, or activity of IL-8, CCL2 (MCP-1), and CCL4 (MIP-1β).

In some embodiments, the immunoconjugate comprises an antibody or antigen-binding fragment thereof that comprises the CDRs of huCD37-3 (i.e., SEQ ID NOs: 4-9). In some embodiments, the antibody is CD37-3. In some embodiments, the immunoconjugate comprises a maytansinoid. In some embodiments, the maytansinoid is DM1. In some embodiments, the immunoconjugate comprises a linker that is SMCC. In some embodiments, the immunoconjugate is IMGN529.

In some embodiments, the anti-CD37 binding agent (e.g., CD37-3-SMCC-DM1) is administered at a dose of about 0.1 mg of anti-CD37 binding agent per kg of body weight (mg/kg) to about 3 mg/kg. In some embodiments, the anti-CD37 binding agent (e.g., CD37-3-SMCC-DM1) is administered at a dose of about 1 mg/kg to about 3 mg/kg. In some embodiments, the anti-CD37 binding agent (e.g., CD37-3-SMCC-DM1) is administered at a dose of about 1 mg/kg to about 2.8 mg/kg. In some embodiments, the anti-CD37 binding agent (e.g., CD37-3-SMCC-DM1) is administered at a dose of about 0.4 mg/kg to about 0.8 mg/kg. In some embodiments, the anti-CD37 binding agent (e.g., CD37-3-SMCC-DM1) is administered at a dose of about 0.8 mg/kg to about 1.4 mg/kg. In some embodiments, the anti-CD37 binding agent (e.g., CD37-3-SMCC-DM1) is administered at a dose of about 1 mg/kg to about 1.4 mg/kg. In some embodiments, the anti-CD37 binding agent (e.g., CD37-3-SMCC-DM1) is administered at a dose of about 1.4 mg/kg to about 2 mg/kg. In some embodiments, the anti-CD37 binding agent (e.g., CD37-3-SMCC-DM1) is administered at a dose of about 1.4 mg/kg to about 3 mg/kg. In some embodiments, the anti-CD37 binding agent (e.g., CD37-3-SMCC-DM1) is administered at a dose of about 1.4 mg/kg to about 2.8 mg/kg. In some embodiments, the anti-CD37 binding agent (e.g., CD37-3-SMCC-DM1) is administered at a dose of about 2 mg/kg to about 2.8 mg/kg. In some embodiments, the anti-CD37 binding agent (e.g., CD37-3-SMCC-DM1) is administered at a dose of about 2 mg/kg to about 3 mg/kg.

According to the methods described herein, the anti-CD37 binding agent (e.g., CD37-3-SMCC-DM1) can be administered about once every 3 weeks. In some embodiments, the anti-CD37 binding agent (e.g., CD37-3-SMCC-DM1) is administered on day 1 of a 21 day cycle.

In some embodiments, the anti-CD37 binding agent (e.g., CD37-3-SMCC-DM1) is administered intravenously.

The methods described herein can be used to treat cancer. In certain embodiments, the cancer is a B-cell malignancy. In certain embodiments, the cancer is a leukemia or lymphoma. In some embodiments, the cancer is selected from the group consisting of B-cell lymphomas including NHL, precursor B-cell lymphoblastic leukemia/lymphoma and mature B-cell neoplasms, such as B-cell chronic lymphocytic leukemia (CLL)/small lymphocytic lymphoma (SLL), B-cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B-cell lymphoma (MALT type, nodal and splenic type), hairy cell leukemia, diffuse large B-cell lymphoma (DLBCL), Burkitt's lymphoma, plasmacytoma, plasma cell myeloma, post-transplant lymphoproliferative disorder, Waldenstrom's macroglobulinemia, and anaplastic large-cell lymphoma (ALCL). In some embodiments, the cancer is relapsed or refractory NHL. In some embodiments, the cancer expresses CD37 polypeptide or nucleic acid. In some embodiments, the subject has already received treatment with an anti-CD20 therapy. In some embodiments, the anti-CD20 therapy includes treatment with an anti-CD20 antibody (e.g., Rituximab).

In some embodiments, the methods further comprise administering a corticosteroid to the patient. In some embodiments, the corticosteroid can be dexamethasone. In some embodiments, the corticosteroid can be administered as a pre-treatment, i.e., prior to the administration of the anti-CD37 binding agent. In some embodiments, the corticosteroid can be administered pre-infusion, during infusion or after infusion or any combination thereof (e.g., peri-infusional). In some embodiments, the corticosteroid can be administered during the administration of the anti-CD37 binding agent.

In some embodiments, the methods further comprise administering a growth factor to the patient. In some embodiments, the growth factor can be granulocyte colony-stimulating factor (G-CSF). In some embodiments, the growth factor can be administered as a pre-treatment, i.e., prior to the administration of the anti-CD37 immunoconjugate. In some embodiments, the growth factor can be administered early to mid-cycle of a 21-day cycle. In some embodiments, the growth factor can be administered about 1 to about 14 days after administration of the anti-CD37 immunoconjugate. In some embodiments, the growth factor can be administered about 1 to about 2 days after administration of the anti-CD37 immunoconjugate. In some embodiments, the growth factor can be administered on day 2 or 3 of a 21-day cycle. In some embodiments, the growth factor can be administered about 5 to about 14 days after administration of the anti-CD37 immunoconjugate. In some embodiments, the growth factor can be administered on at least one day from day 6 to day 15 of a 21-day cycle. In some embodiments, the growth factor can be administered from day 14 to day 21 of a 21-day cycle.

In some embodiments, the administration of corticosteroids and/or G-CSF to the dosing protocol allows a higher dose to be administered, longer duration of treatment, less neutropenia, and/or more clinical benefit.

The methods described herein can result in a decrease in tumor burden. The methods described herein can also result in a decrease in adverse effects. For example, administration of an anti-CD37 immunoconjugate in combination with a corticosteroid and a growth factor (e.g., G-CSF), can prevent the occurrence, decrease the severity, and/or shorten the duration of neutropenia.

The methods described herein can result in a decrease, shortening, or prevention of neutropenia. The methods described herein can result in a decrease in the likelihood of neutropenia. The methods described herein can result in a decrease in the severity of neutropenia.

### BRIEF DESCRIPTION OF THE DRAWINGS/FIGURES

FIG. 1 provides absolute neutrophil counts (ANC) of patients treated with varying doses of IMGN529, in the absence of peri-infusional corticosteroid treatment.

FIG. 2 provides lymphocyte levels of patients treated with varying doses of IMGN529, in the absence of peri-infusional corticosteroid treatment.

FIG. 3 provides absolute neutrophil counts (ANC) of patients treated with varying doses of IMGN529 and peri-infusional corticosteroid treatment.

FIG. 4 provides lymphocyte levels of patients treated with varying doses of IMGN529 and peri-infusional corticosteroid treatment.

FIG. 5 provides absolute neutrophil counts (ANC) of patients treated with varying doses of IMGN529 and peri-infusional corticosteroid treatment, by Cycle and Day (C#D#).

FIG. 6 provides lymphocyte levels of patients treated with varying doses of IMGN529 and peri-infusional corticosteroid treatment, by Cycle and Day (C#D#).

FIGS. 7A and 7B provide pharmacokinetic data obtained from patients treated with IMGN529. FIG. 7A shows the plasma concentration of IMGN529 in individual patients over time, and FIG. 7B shows the average plasma concentration of IMGN529 over time.

FIG. 8 provides CD37 prevalence data in B-cell non-Hodgkin lymphoma (NHL) and chronic lymphocytic leukemia (CLL).

FIG. 9 provides sample patient baseline characteristics.

FIG. 10 provides initial dose escalation details and observed dose-limiting toxicities (DLTs).

FIGS. 11A and 11B provide dose re-escalation with peri-infusional corticosteroids and observed DLTs.

FIG. 12 provides an overview of patients with treatment emergent adverse events (TEAEs). Grade 4 AEs were reported in the 0.7, 0.8 and 1.0 mg/kg dose cohorts: Neutropenia (N=3), Hypocalcaemia (N=1). Grade 5 AE: Cardiac Arrest, Unrelated (0.7 mg/kg N=1). Most common related AEs experienced by more than three patients includes all AE terms listed above excluding hypokalaemia and hypotension.

FIG. 13 provides a graph showing the weeks on treatment for each patient in each dosing category. “C1D1” indicates Cycle 1, Day 1. “A3” indicates patients who received G-CSF in their first cycle (other patients received G-CSF in later cycles).

FIG. 14A depicts CD37 expression in antibodies bound to cells (ABC) values in normal human peripheral blood cells and in vitro depletion of CD19+B cells and CD66b+ granulocytes or neutrophils after 1 hour or 20 hours treatment with IMGN529, rituximab (an anti-CD20 antibody), alemtuzumab (an anti-CD52 antibody) or a non-specific IgG1-SMCC-DM1 control conjugate. FIG. 14B depicts cytokine levels in cell culture supernatants for IL-8, IL-6, CCL2 (MCP-1), and CCL4 (MIP-113) after treatment of normal human peripheral blood cells with indicated agents for approximately 20-24 hours.

FIGS. 15A and 15B depict percent changes in absolute lymphocyte counts (ALC) and absolute neutrophil counts (ANC) at day 2, day 5, and day 14 of treatment with (A) 1, 3, and 10 mg/kg muCD37-ADC or 10 mg/kg of a non-specific IgG1-SMCC-DM1 control conjugate, shown in FIG. 15A; or (B) 10 mg/kg muCD37 antibody, muCD37-ADC, IgG1-SMCC-DM1 control conjugate, or anti-Ly6G antibody, shown in FIG. 15B. FIG. 15C depicts cytokine levels in mouse plasma after treatment with 10 mg/kg muCD37-ADC, IgG1-SMCC-DM1 control conjugate, or anti-Ly6G antibody.

FIG. 16A depicts absolute lymphocyte counts (ALC) and absolute neutrophil counts (ANC) before and after treatment with muCD37-ADC, IgG1-SMCC-DM1 control conjugate, or cyclophosphamide (CPA). FIG. 16B depicts percentage of various precursors in bone marrow smears on day 2 following treatment with muCD37-ADC, IgG1-SMCC-DM1 control conjugate, or CPA.

### DETAILED DESCRIPTION OF THE INVENTION

The present invention provides new dosing regimens for CD37 binding immunoconjugates.

### I. DEFINITIONS

To facilitate an understanding of the present invention, a number of terms and phrases are defined below.

The term “CD37” as used herein, refers to any native CD37, unless otherwise indicated. CD37 is also referred to as GP52-40, leukocyte antigen CD37, and Tetraspanin-26. The term “CD37” encompasses “full-length,” unprocessed CD37 as well as any form of CD37 that results from processing in the cell. The term also encompasses naturally occurring variants of CD37, e.g., splice variants, allelic variants, and isoforms. The CD37 polypeptides described herein can be isolated from a variety of sources, such as from human tissue types or from another source, or prepared by recombinant or synthetic methods.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

A “blocking” antibody or an “antagonist” antibody is one which inhibits or reduces biological activity of the antigen it binds, such as CD37. In some embodiments, blocking antibodies or antagonist antibodies substantially or completely inhibit the biological activity of the antigen. The biological activity can be reduced by 10%, 20%, 30%, 50%, 70%, 80%, 90%, 95%, or even 100%.

The term “anti-CD37 antibody” or “an antibody that binds to CD37” refers to an antibody that is capable of binding CD37 with sufficient affinity such that the antibody is useful as a diagnostic and/or therapeutic agent in targeting CD37. The extent of binding of an anti-CD37 antibody to an unrelated, non-CD37 protein can be less than about 10% of the binding of the antibody to CD37 as measured, e.g., by a radioimmunoassay (RIA). In certain embodiments, an antibody that binds to CD37 has a dissociation constant (Kd) of ≦1 μM, ≦100 nM, ≦10 nM, ≦1 nM, or ≦0.1 nM.

The term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, single chain antibodies, and multispecific antibodies formed from antibody fragments.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “humanized antibody” refers to forms of non-human (e.g. murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g. mouse, rat, rabbit, hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FR) residues of a human immunoglobulin are replaced with the corresponding residues in an antibody from a non-human species that has the desired specificity, affinity, and capability. The humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539, Roguska et al., Proc. Natl. Acad. Sci., USA, 91(3):969-973 (1994), and Roguska et al., Protein Eng. 9(10):895-904 (1996). In some embodiments, a “humanized antibody” is a resurfaced antibody.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al (1997) J. Molec. Biol. 273:927-948)). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g, Kabat et al., Sequences of Immunological Interest. 5th Ed. Public Health Service, National Institutes of Health, Bethesda, Md. (1991)).

The amino acid position numbering as in Kabat, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g. mouse, rat, rabbit, etc) with the desired specificity, affinity, and capability while the constant regions are homologous to the sequences in antibodies derived from another (usually human) to avoid eliciting an immune response in that species.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

“Or better” when used herein to refer to binding affinity refers to a stronger binding between a molecule and its binding partner. “Or better” when used herein refers to a stronger binding, represented by a smaller numerical Kd value. For example, an antibody which has an affinity for an antigen of “0.6 nM or better”, the antibody's affinity for the antigen is <0.6 nM, i.e. 0.59 nM, 0.58 nM, 0.57 nM etc. or any value less than 0.6 nM.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

An antibody is said to “competitively inhibit” binding of a reference antibody to a given epitope if it preferentially binds to that epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

The phrase “substantially similar,” or “substantially the same”, as used herein, denotes a sufficiently high degree of similarity between two numeric values (generally one associated with an antibody of the invention and the other associated with a reference/comparator antibody) such that one of skill in the art would consider the difference between the two values to be of little or no biological and/or statistical significance within the context of the biological characteristic measured by said values (e.g., Kd values). The difference between said two values can be less than about 50%, less than about 40%, less than about 30%, less than about 20%, or less than about 10% as a function of the value for the reference/comparator antibody.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure.

As used herein, “substantially pure” refers to material which is at least 50% pure (i.e., free from contaminants), at least 90% pure, at least 95% pure, at least 98% pure, or at least 99% pure.

The term “immunoconjugate” or “conjugate” as used herein refers to a compound or a derivative thereof that is linked to a cell binding agent (i.e., an anti-CD37 antibody or fragment thereof) and is defined by a generic formula: C-L-A, wherein C=cytotoxin, L=linker, and A=anti-CD37 antibody or antibody fragment Immunoconjugates can also be defined by the generic formula in reverse order: A-L-C.

The term “IMGN529” refers to the immunoconjugate described herein containing the huCD37-3 antibody (comprising the CDRs represented by SEQ ID NOs: 4-9, the VH of SEQ ID NO:12 and the VL of SEQ ID NO:15), the SMCC linker, and the DM1 maytansinoid.

A “linker” is any chemical moiety that is capable of linking a compound, usually a drug, such as a maytansinoid, to a cell-binding agent such as an anti CD37 antibody or a fragment thereof in a stable, covalent manner Linkers can be susceptible to or be substantially resistant to acid-induced cleavage, light-induced cleavage, peptidase-induced cleavage, esterase-induced cleavage, and disulfide bond cleavage, at conditions under which the compound or the antibody remains active. Suitable linkers are well known in the art and include, for example, disulfide groups, thioether groups, acid labile groups, photolabile groups, peptidase labile groups and esterase labile groups. Linkers also include charged linkers, and hydrophilic forms thereof as described herein and know in the art.

The terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. “Tumor” and “neoplasm” refer to one or more cells that result from excessive cell growth or proliferation, either benign (noncancerous) or malignant (cancerous) including pre-cancerous lesions. Examples of “cancer” or “tumorigenic” diseases which can be treated and/or prevented include B-cell lymphomas including NHL, precursor B-cell lymphoblastic leukemia/lymphoma and mature B-cell neoplasms, such as B-cell chronic lymphocytic leukemia (CLL)/small lymphocytic lymphoma (SLL), B-cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B-cell lymphoma (MALT type, nodal and splenic type), hairy cell leukemia, diffuse large B-cell lymphoma, Burkitt's lymphoma, plasmacytoma, plasma cell myeloma, post-transplant lymphoproliferative disorder, Waldenstrom's macroglobulinemia, and anaplastic large-cell lymphoma (ALCL).

The terms “cancer cell,” “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

Administration “in combination with” one or more further therapeutic agents includes simultaneous (concurrent) and consecutive administration in any order.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

An “effective amount” of an antibody or immunoconjugate as disclosed herein is an amount sufficient to carry out a specifically stated purpose. An “effective amount” can be determined empirically and in a routine manner, in relation to the stated purpose.

The term “therapeutically effective amount” refers to an amount of an antibody or other drug effective to “treat” a disease or disorder in a subject or mammal. In the case of cancer, the therapeutically effective amount of the drug can reduce the number of cancer cells; reduce the tumor size or burden; inhibit (i.e., slow to some extent and in a certain embodiment, stop) cancer cell infiltration into peripheral organs; inhibit (i.e., slow to some extent and in a certain embodiment, stop) tumor metastasis; inhibit, to some extent, tumor growth; relieve to some extent one or more of the symptoms associated with the cancer; and/or result in a favorable response such as increased progression-free survival (PFS), disease-free survival (DFS), or overall survival (OS), complete response (CR), partial response (PR), or, in some cases, stable disease (SD), a decrease in progressive disease (PD), a reduced time to progression (TTP) or any combination thereof. See the definition herein of “treating”. To the extent the drug can prevent growth and/or kill existing cancer cells, it can be cytostatic and/or cytotoxic. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

A “chemotherapeutic agent” is a chemical compound useful in the treatment of cancer, regardless of mechanism of action. Chemotherapeutic agents include, for example, antagonists of CD20 such as Rituximab and cyclophosphamide, doxorubicin, vincristine, predinisone, fludarabine, etoposide, methotrexate, lenalidomide, chlorambucil, bentamustine and/or modified versions of such chemotherapeutics.

The term “respond favorably” generally refers to causing a beneficial state in a subject. With respect to cancer treatment, the term refers to providing a therapeutic effect on the subject. Positive therapeutic effects in cancer can be measured in a number of ways (See, W. A. Weber, J. Nucl. Med. 50:1S-10S (2009)). For example, tumor growth inhibition, molecular marker expression, serum marker expression, and molecular imaging techniques can all be used to assess therapeutic efficacy of an anti-cancer therapeutic. With respect to tumor growth inhibition, according to NCI standards, a T/C≦42% is the minimum level of anti-tumor activity. A T/C<10% is considered a high anti-tumor activity level, with T/C (%)=Median tumor volume of the treated/Median tumor volume of the control×100. A favorable response can be assessed, for example, by increased progression-free survival (PFS), disease-free survival (DFS), or overall survival (OS), complete response (CR), partial response (PR), or, in some cases, stable disease (SD), a decrease in progressive disease (PD), a reduced time to progression (TTP) or any combination thereof.

PFS, DFS, and OS can be measured by standards set by the National Cancer Institute and the U.S. Food and Drug Administration for the approval of new drugs. See Johnson et al, (2003) J. Clin. Oncol. 21(7):1404-1411.

“Progression free survival” (PFS) refers to the time from enrollment to disease progression or death. PFS is generally measured using the Kaplan-Meier method and Response Evaluation Criteria in Solid Tumors (RECIST) 1.1 standards. Generally, progression free survival refers to the situation wherein a patient remains alive, without the cancer getting worse.

“Time to Tumor Progression” (TTP) is defined as the time from enrollment to disease progression. TTP is generally measured using the RECIST 1.1 criteria.

A “complete response” or “complete remission” or “CR” indicates the disappearance of all signs of tumor or cancer in response to treatment. This does not always mean the cancer has been cured.

A “partial response” or “PR” refers to a decrease in the size or volume of one or more tumors or lesions, or in the extent of cancer in the body, in response to treatment.

“Stable disease” refers to disease without progression or relapse. In stable disease there is neither sufficient tumor shrinkage to qualify for partial response nor sufficient tumor increase to qualify as progressive disease.

“Progressive disease” refers to the appearance of one more new lesions or tumors and/or the unequivocal progression of existing non-target lesions. Progressive disease can also refer to a tumor growth of more than 20 percent since treatment began, either due to an increases in mass or in spread of the tumor.

“Disease free survival” (DFS) refers to the length of time during and after treatment that the patient remains free of disease.

“Overall Survival” (OS) refers to the time from patient enrollment to death or censored at the date last known alive. OS includes a prolongation in life expectancy as compared to naive or untreated individuals or patients. Overall survival refers to the situation wherein a patient remains alive for a defined period of time, such as one year, five years, etc., e.g., from the time of diagnosis or treatment.

The term “overexpression” of CD37 in a particular tumor, tissue, or cell sample refers to CD37 (a CD37 polypeptide or a nucleic acid encoding such a polypeptide) that is present at a level higher than that which is present in non-diseased tissue or cells of the same type or origin. Such overexpression can be caused, for example, by mutation, gene amplification, increased transcription, or increased translation.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. In certain embodiments, a subject is successfully “treated” for cancer according to the methods of the present invention if the patient shows one or more of the following: a reduction in the number of or complete absence of cancer cells; a reduction in the tumor burden; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), or overall survival (OS), complete response (CR), partial response (PR), stable disease (SD), a decrease in progressive disease (PD), a reduced time to progression (TTP), or any combination thereof.

Prophylactic or preventative measures refer to measures that prevent and/or slow the development of a targeted pathological condition or disorder. Thus, those in need of prophylactic or preventative measures include those prone to have the disorder and those in whom the disorder is to be prevented.

The terms “pre-treat” and “pre-treatment” refer to therapeutic measures that occur prior to the administration of an anti-CD37 therapeutic. For example, as described in more detail herein, a prophylactic such as a steroid (e.g., corticosteroid) can be administered within about a week, about five days, about three days, about two days, or about one day or 24 hours prior to the administration of the anti-CD37 therapeutic. The prophylactic can also be administered prior to the anti-CD37 therapeutic on the same day as the anti-CD37 therapeutic.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in Karlin et al, 1990, Proc. Natl. Acad. Sci., 87:2264-2268, as modified in Karlin et al., 1993, Proc. Natl. Acad. Sci., 90:5873-5877, and incorporated into the NBLAST and XBLAST programs (Altschul et al., 1991, Nucleic Acids Res., 25:3389-3402). In certain embodiments, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, Calif.) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48):444-453 (1970)) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be longer than the percent identity of the second sequence to the first sequence.

As a non-limiting example, whether any particular polynucleotide has a certain percentage sequence identity (e.g., is at least 80% identical, at least 85% identical, at least 90% identical, and in some embodiments, at least 95%, 96%, 97%, 98%, or 99% identical) to a reference sequence can, in certain embodiments, be determined using the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2: 482 489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. Identity can exist over a region of the sequences that is at least about 10, about 20, about 40-60 residues in length or any integral value there between, and can be over a longer region than 60-80 residues, for example, at least about 90-100 residues, and in some embodiments, the sequences are substantially identical over the full length of the sequences being compared, such as the coding region of a nucleotide sequence for example.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In some embodiments, conservative substitutions in the sequences of the polypeptides and antibodies of the invention do not abrogate the binding of the polypeptide or antibody containing the amino acid sequence, to the antigen(s), i.e., the CD37 to which the polypeptide or antibody binds. Methods of identifying nucleotide and amino acid conservative substitutions which do not eliminate antigen binding are well-known in the art (see, e.g., Brummell et al., Biochem. 32: 1180-1 187 (1993); Kobayashi et al. Protein Eng. 12(10):879-884 (1999); and Burks et al. Proc. Natl. Acad. Sci. USA 94:412-417 (1997)).

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. CD37-BINDING AGENTS

The methods described herein provide methods of administering agents that specifically bind CD37. These agents are referred to herein as “CD37-binding agents.” The full-length amino acid sequences for human, macaque, and murine CD37 are known in the art and also provided herein as represented by SEQ ID NOs: 1-3, respectively.

Human CD37:

(SEQ ID NO: 1)

MSAQESCLSLIKYFLFVFNLFFFVLGSLIFCFGIWILIDKTSFVSFVGLA
FVPLQIWSKVLAISGIFTMGIALLGCVGALKELRCLLGLYFGMLLLLFAT
QITLGILISTQRAQLERSLRDVVEKTIQKYGTNPEETAAEESWDYVQFQL
RCCGWHYPQDWFQVLILRGNGSEAHRVPCSCYNLSATNDSTILDKVILPQ
LSRLGHLARSRHSADICAVPAESHIYREGCAQGLQKWLHNNLISIVGICL
GVGLLELGFMTLSIFLCRNLDHVYNRLAYR
Macaque CD37:

(SEQ ID NO: 2)

MSAQESCLSLIKYFLFVFNLFFFVILGSLIFCFGIWILIDKTSFVSFVGL
AFVPLQIWSKVLAISGVFTMGLALLGCVGALKELRCLLGLYFGMLLLLFA
TQITLGILISTQRAQLERSLQDIVEKTIQRYHTNPEETAAEESWDYVQFQ
LRCCGWHSPQDWFQVLTLRGNGSEAHRVPCSCYNLSATNDSTILDKVILP
QLSRLGQLARSRHSTDICAVPANSHIYREGCARSLQKWLHNNLISIVGIC
LGVGLLELGFMTLSIFLCRNLDHVYNRLRYR
Murine CD37 (NP_031671):

(SEQ ID NO: 3)

MSAQESCLSLIKYFLFVFNLFFFVLGGLIFCFGTWILIDKTSFVSFVGLS
FVPLQTWSKVLAVSGVLTMALALLGCVGALKELRCLLGLYFGMLLLLFAT
QITLGILISTQRVRLERRVQELVLRTIQSYRTNPDETAAEESWDYAQFQL
RCCGWQSPRDWNKAQMLKANESEEPFVPCSCYNSTATNDSTVFDKLFFSQ
LSRLGPRAKLRQTADICALPAKAHIYREGCAQSLQKWLHNNIISIVGICL
GVGLLELGFMTLSIFLCRNLDHVYDRLARYR

In certain embodiments, the CD37-binding agents are antibodies, immunoconjugates or polypeptides. In some embodiments, the CD37-binding agents are humanized antibodies.

In certain embodiments, the CD37-binding agents have one or more of the following effects: inhibit proliferation of tumor cells, reduce the tumorigenicity of a tumor by reducing the frequency of cancer stem cells in the tumor, inhibit tumor growth, increase survival, trigger cell death of tumor cells, differentiate tumorigenic cells to a non-tumorigenic state, or prevent metastasis of tumor cells.

In certain embodiments, immunoconjugates or other agents that specifically bind human CD37 trigger cell death via a cytotoxic agent. For example, in certain embodiments, an antibody to a human CD37 antibody is conjugated to a maytansinoid that is activated in tumor cells expressing the CD37 by protein internalization. In certain alternative embodiments, the agent or antibody is not conjugated.

In certain embodiments, the CD37-binding agents are capable of inhibiting tumor growth. In certain embodiments, the CD37-binding agents are capable of inhibiting tumor growth in vivo (e.g., in a xenograft mouse model and/or in a human having cancer).

The CD37-binding agents include the antibody huCD37-3 and fragments, variants and derivatives thereof, as described previously in U.S. Publication No. 2011/0256153, which is herein incorporated by reference in its entirety. The CD37-binding agents also include CD37-binding agents that specifically bind to the same CD37 epitope as huCD37-3. The CD37-binding agents also include CD37-binding agents that competitively inhibit huCD37-3.

The CD37-binding agents also include CD37-binding agents that comprise the heavy and light chain CDR sequences of huCD37-3. The CDR sequences of huCD37-3 are described in Tables 1 and 2 below.

TABLE 1
Variable heavy chain CDR amino acid sequences

| Antibody | VH-CDR1 | VH-CDR2 | VH-CDR3 |
|---|---|---|---|
| CD37-3 | TSGVS | VIWGDGSTN | GGYSLAH |
| | (SEQ ID NO: | (SEQ ID NO: 5) | (SEQ ID NO: 6) |
| | 4) | | |

TABLE 2
Variable light chain CDR amino acid sequences

| Antibody | VL-CDR1 | VL-CDR2 | VL-CDR3 |
|---|---|---|---|
| CD37-3 | RASENIRSNLA | VATNLAD | QHYWGTTWT |
| | (SEQ ID NO: | (SEQ ID NO: 8) | (SEQ ID NO: 9) |
| | 7) | | |

The CD37 binding molecules can be antibodies or antigen binding fragments that specifically bind to CD37 that comprise the CDRs of murine, chimeric, or humanized CD37-3 with up to four (i.e. 0, 1, 2, 3, or 4) conservative amino acid substitutions per CDR. In some embodiments, the CD37-binding agents comprise variable heavy chain CDR1, CDR2, and CDR3 sequences comprising SEQ ID NOs: 4, 5, and 6 and variable light chain CDR1, CDR2, and CDR3 sequences comprising SEQ ID NOs: 7, 8, and 9.

Polypeptides can comprise the variable light chains or variable heavy chains described herein. Antibodies and polypeptides can also comprise both a variable light chain and a variable heavy chain. The variable light chain and variable heavy chain sequences of murine, chimeric, and humanized CD37-3 antibodies are provided in Tables 3 and 4 below.

TABLE 3
Variable heavy chain amino acid sequences

| Antibody | VH Amino Acid Sequence (SEQ ID NO) |
|---|---|
| muCD37-3 | QVQVKESGPGLVAPSQSLSITCTVSGFSLTTSGVSWVRQPPGKGLEWLGVIW |
| | GDGSTNYHSALKSRLSIKKDHSKSQVFLKLNSLQTDDTATYYCAKGGYSLA |
| | HWGQGTLVTVSA (SEQ ID NO: 10) |
| chCD37-3 | QVQVKESGPGLVAPSQSLSITCTVSGFSLTTSGVSWVRQPPGKGLEWLGVIW |
| | GDGSTNYHSALKSRLSIKKDHSKSQVFLKLNSLQTDDTATYYCAKGGYSLA |
| | HWGQGTLVTVSA (SEQ ID NO: 11) |
| huCD37-3 | QVQVQESGPGLVAPSQTLSITCTVSGFSLTTSGVSWVRQPPGKGLEWLGVIW |
| (version 1.0) | GDGSTNYHPSLKSRLSIKKDHSKSQVFLKLNSLTAADTATYYCAKGGYSLA |
| | HWGQGTLVTVSS (SEQ ID NO: 12) |
| huCD37-3 | QVQVQESGPGLVAPSQTLSITCTVSGFSLTTSGVSWVRQPPGKGLEWLGVIW |
| (version 1.1) | GDGSTNYHSSLKSRLSIKKDHSKSQVFLKLNSLTAADTATYYCAKGGYSLA |
| | HWGQGTLVTVSS (SEQ ID NO: 22) |

TABLE 4
Variable light chain amino acid sequences

| Antibody | VL Amino Acid Sequence (SEQ ID NO) |
|---|---|
| muCD37-3 | DIQMTQSPASLSVSVGETVTITCRASENIRSNLAWYQQKQGKSPQLLVNVAT |
| | NLADGVPSRFSGSGSGTQYSLKINSLQSEDFGTYYCQHYWGTTWTFGGGTK |
| | LEIKR (SEQ ID NO: 13) |
| chCD37-3 | DIQMTQSPASLSVSVGETVTITCRASENIRSNLAWYQQKQGKSPQLLVNVAT |
| | NLADGVPSRFSGSGSGTQYSLKINSLQSEDFGTYYCQHYWGTTWTFGGGTK |
| | LEIKR (SEQ ID NO: 14) |
| huCD37-3 | DIQMTQSPSSLSVSVGERVTITCRASENIRSNLAWYQQKPGKSPKLLVNVAT |
| | NLADGVPSRFSGSGSGTDYSLKINSLQPEDFGTYYCQHYWGTTWTFGQGTK |
| | LEIKR (SEQ ID NO: 15) |

Also provided are antibodies and antigen-binding fragments thereof that comprise: (a) a VH polypeptide having at least about 90% sequence identity to one of SEQ ID NOs: 10-12 and 22; and/or (b) a VL polypeptide having at least about 90% sequence identity to one of SEQ ID NOs: 13-15. In certain embodiments, the antibody or antigen-binding fragment thereof comprises (a) a VH polypeptide having at least about 95%, at least about 96%, at least about 97%, at least about 98%, or at least about 99% sequence identity to one of SEQ ID NOs: 10-12 and 22 and (b) a VL polypeptide having at least about 95%, at least about 96%, at least about 97%, at least about 98%, or at least about 99% sequence identity to one of SEQ ID NOs: 13-15. In certain embodiments, the antibody or antigen-binding fragment thereof comprises (a) a VH polypeptide having at least about 95% sequence identity to one of SEQ ID NOs: 10-12 and 22, and (b) a VL polypeptide having at least about 95% sequence identity to one of SEQ ID NOs: 13-15. In certain embodiments, the antibody or antigen-binding fragment comprises (a) a VH polypeptide having the amino acid sequence of one of SEQ ID NOs: 10-12 and 22; and (b) a VL polypeptide having the amino acid sequence of one of SEQ ID NOs: 13-15. In certain embodiments, the antibody or antigen-binding fragment specifically binds CD37. In certain embodiments, the antibody or antigen-binding fragment is a murine, chimeric, or humanized antibody that specifically binds CD37. In certain embodiments, the antibody or antigen-binding fragment containing polypeptides having a certain percentage of sequence identity to SEQ ID NOs: 10-12 and 22 and 13-15 differs from SEQ ID NOs: 10-12 and 13-15 by conservative amino acid substitutions only.

Antibodies and antigen-binding fragments thereof can also comprise both a light chain and a heavy chain. The light chain and variable chain sequences of murine, chimeric, and humanized CD37-3 antibodies are provided in Tables 5 and 6 below.

TABLE 5
Full-length heavy chain amino acid sequences

| Antibody | Full-Length Heavy Chain Amino Acid Sequence (SEQ ID NO) |
|---|---|
| muCD37-3 | QVQVKESGPGLVAPSQSLSITCTVSGFSLTTSGVSWVRQPPGKGLEWLGVIW |
| | GDGSTNYHSALKSRLSIKKDHSKSQVFLKLNSLQTDDTATYYCAKGGYSLA |
| | HWGQGTLVTVSAAKTTAPSVYPLAPVCGDTTGSSVTLGCLVKGYFPEPVTL |
| | TWNSGSLSSGVHTFPAVLQSDLYTLSSSVTVTSSTWPSQSITCNVAHPASSTK |
| | VDKKIEPRGPTIKPCPPCKCPAPNLLGGPSVFIFPPKIKDVLMISLSPIVTCVVV |
| | DVSEDDPDVQISWFVNNVEVHTAQTQTHREDYNSTLRVVSALPIQHQDWM |
| | SGKEFKCKVNNKDLPAPIERTISKPKGSVRAPQVYVLPPPEEEMTKKQVTLT |
| | CMVTDFMPEDIYVEWTNNGKTELNYKNTEPVLDSDGSYFMYSKLRVEKKN |
| | WVERNSYSCSVVHEGLHNHHTTKSFSRTPGK (SEQ ID NO: 16) |
| chCD37-3 | QVQVKESGPGLVAPSQSLSITCTVSGFSLTTSGVSWVRQPPGKGLEWLGVIW |
| | GDGSTNYHSALKSRLSIKKDHSKSQVFLKLNSLQTDDTATYYCAKGGYSLA |
| | HWGQGTLVTVSAASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNT |
| | KVDKKVEPKSCDKTHTCPPCPAPELLGGPSVFLFPPKPKDTLMISRTPEVTCV |
| | VVDVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSRDELTKNQ |
| | VSLTCLVKGFYPSDIAVEWESNGQPENNYKTTPPVLDSDGSFFLYSKLTVDK |
| | SRWQQGNVFSCSVMHEALHNHYTQKSLSLSPGK (SEQ ID NO: 17) |
| huCD37-3Q | VQVQESGPGLVAPSQTLSITCTVSGFSLTTSGVSWVRQPPGKGLEWLGVIW |
| | GDGSTNYHPSLKSRLSIKKDHSKSQVFLKLNSLTAADTATYYCAKGGYSLA |
| | HWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNT |
| | KVDKKVEPKSCDKTHTCPPCPAPELLGGPSVFLFPPKPKDTLMISRTPEVTCV |
| | VVDVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSRDELTKNQ |
| | VSLTCLVKGFYPSDIAVEWESNGQPENNYKTTPPVLDSDGSFFLYSKLTVDK |
| | SRWQQGNVFSCSVMHEALHNHYTQKSLSLSPG (SEQ ID NO: 18) |

TABLE 6
Full-length light chain amino acid sequences

| Antibody | Full-length Light Chain Amino Acid Sequence (SEQ ID NO) |
|---|---|
| muCD37-3 | DIQMTQSPASLSVSVGETVTITCRASENIRSNLAWYQQKQGKSPQLLVNVAT |
| | NLADGVPSRFSGSGSGTQYSLKINSLQSEDFGTYYCQHYWGTTWTFGGGTK |
| | LEIKRADAAPTVSIFPPSSEQLTSGGASVVCFLNNFYPKDINVKWKIDGSERQ |
| | NGVLNSWTDQDSKDSTYSMSSTLTLTKDEYERHNSYTCEATHKTSTSPIVKS |
| | FNRNEC (SEQ ID NO: 19) |
| chCD37-3 | DIQMTQSPASLSVSVGETVTITCRASENIRSNLAWYQQKQGKSPQLLVNVAT |
| | NLADGVPSRFSGSGSGTQYSLKINSLQSEDFGTYYCQHYWGTTWTFGGGTK |
| | LEIKRTVAAPSVFIFPPSDEQLKSGTASVVCLLNNFYPREAKVQWKVDNALQ |
| | SGNSQESVTEQDSKDSTYSLSSTLTLSKADYEKHKVYACEVTHQGLSSPVTK |
| | SFNRGEC (SEQ ID NO: 20) |
| huCD37-3 | DIQMTQSPSSLSVSVGERVTITCRASENIRSNLAWYQQKPGKSPKLLVNVAT |
| | NLADGVPSRFSGSGSGTDYSLKINSLQPEDFGTYYCQHYWGTTWTFGQGTK |
| | LEIKRTVAAPSVFIFPPSDEQLKSGTASVVCLLNNFYPREAKVQWKVDNALQ |
| | SGNSQESVTEQDSKDSTYSLSSTLTLSKADYEKHKVYACEVTHQGLSSPVTK |
| | SFNRGEC (SEQ ID NO: 21) |

Also provided are antibodies and antigen-binding fragments thereof that comprise: (a) a polypeptide having at least about 90% sequence identity to one of SEQ ID NOs: 16-18; and (b) a polypeptide having at least about 90% sequence identity to one of SEQ ID NOs: 19-21. In certain embodiments, the antibody or antigen-binding fragment thereof comprises a polypeptide having at least about 95%, at least about 96%, at least about 97%, at least about 98%, or at least about 99% sequence identity to one of SEQ ID NOs: 16-18 and a polypeptide having at least about 95%, at least about 96%, at least about 97%, at least about 98%, or at least about 99% sequence identity to one of SEQ ID NOs: 19-21. Thus, in certain embodiments, the antibody or antigen-binding fragment comprises (a) a polypeptide having at least about 95% sequence identity to one of SEQ ID NOs: 16-18, and/or (b) a polypeptide having at least about 95% sequence identity to one of SEQ ID NOs: 19-21. In certain embodiments, the antibody or antigen-binding fragment comprises (a) a polypeptide having the amino acid sequence of one of SEQ ID NOs: 16-18; and/or (b) a polypeptide having the amino acid sequence of one of SEQ ID NOs: 19-21. In certain embodiments, the antibody or antigen-binding fragment thereof is a murine, chimeric, or humanized antibody or fragment that specifically binds CD37. In certain embodiments, the antibody or antigen-binding fragment thereof comprises polypeptides differing from SEQ ID NOs: 16-18 and 19-21 by conservative amino acid substitutions only.

In certain embodiments, the CD37 antibody can be the antibody produced from a hybridoma selected from the group consisting of consisting of ATCC Deposit Designation PTA-10664, deposited with the ATCC on Feb. 18, 2010. In certain embodiments, the antibody comprises the VH-CDRs and the VL-CDRS of the antibody produced from a hybridoma selected from the group consisting of PTA-10664.

In certain embodiments, the CD37 antibody can comprise a light chain encoded by the recombinant plasmid DNA phuCD37-3LC (ATCC Deposit Designation PTA-10722, deposited with the ATCC on Mar. 18, 2010). In certain embodiments, the CD37 antibody can comprise a heavy chain encoded by the recombinant plasmid DNA phuCD37-3HCv.1.0 (ATCC Deposit Designation PTA-10723, deposited with the ATCC on Mar. 18, 2010). In certain embodiments, the CD37 antibody can comprise a light chain encoded by the recombinant plasmid DNA phuCD37-3LC (PTA-10722) and a heavy chain encoded by the recombinant plasmid DNA phuCD37-3HCv.1.0 (PTA-10723). In certain embodiments, the CD37 antibody can comprise the VL-CDRs encoded by the recombinant plasmid DNA phuCD37-3LC (PTA-10722) and the VH-CDRs encoded by the recombinant plasmid DNA phuCD37-3HCv.1.0 (PTA-10723).

Methods known in the art for purifying antibodies and other proteins also include, for example, those described in U.S. Patent Publication No. 2008/0312425, 2008/0177048, and 2009/0187005, each of which is hereby incorporated by reference herein in its entirety.

### III. IMMUNOCONJUGATES

Methods for administering conjugates comprising the anti-CD37 antibodies, antibody fragments, and their functional equivalents as disclosed herein, linked or conjugated to a drug or prodrug (also referred to herein as immunoconjugates) are also described herein. Suitable drugs or prodrugs are known in the art. The drugs or prodrugs can be cytotoxic agents. The cytotoxic agent used in the cytotoxic conjugate of the present invention can be any compound that results in the death of a cell, or induces cell death, or in some manner decreases cell viability, and includes, for example, maytansinoids and maytansinoid analogs. Other suitable cytotoxic agents are for example benzodiazepines, taxoids, CC-1065 and CC-1065 analogs, duocarmycins and duocarmycin analogs, enediynes, such as calicheamicins, dolastatin and dolastatin analogs including auristatins, tomaymycin derivaties, leptomycin derivaties, methotrexate, cisplatin, carboplatin, daunorubicin, doxorubicin, vincristine, vinblastine, melphalan, mitomycin C, chlorambucil and morpholino doxorubicin.

Such conjugates can be prepared by using a linking group in order to link a drug or prodrug to the antibody or functional equivalent. Suitable linking groups are well known in the art and include, for example, disulfide groups, thioether groups, acid labile groups, photolabile groups, peptidase labile groups and esterase labile groups.

The drug or prodrug can, for example, be linked to the anti-CD37 antibody or fragment thereof through a disulfide bond. The linker molecule or crosslinking agent comprises a reactive chemical group that can react with the anti-CD37 antibody or fragment thereof. The reactive chemical groups for reaction with the cell-binding agent can be N-succinimidyl esters and N-sulfosuccinimidyl esters. Additionally the linker molecule comprises a reactive chemical group, which can be a dithiopyridyl group that can react with the drug to form a disulfide bond. Linker molecules include, for example, N-succinimidyl 3-(2-pyridyldithio) propionate (SPDP) (see, e.g., Carlsson et al., Biochem. J., 173: 723-737 (1978)), N-succinimidyl 4-(2-pyridyldithio)butanoate (SPDB) (see, e.g., U.S. Pat. No. 4,563,304), N-succinimidyl 4-(2-pyridyldithio)2-sulfobutanoate (sulfo-SPDB) (see US Publication No. 20090274713), N-succinimidyl 4-(2-pyridyldithio) pentanoate (SPP) (see, e.g., CAS Registry number 341498-08-6), 2-iminothiolane, or acetylsuccinic anhydride. For example, the antibody or cell binding agent can be modified with crosslinking reagents and the antibody or cell binding agent containing free or protected thiol groups thus derived is then reacted with a disulfide- or thiol-containing maytansinoid to produce conjugates. The conjugates can be purified by chromatography, including but not limited to HPLC, size-exclusion, adsorption, ion exchange and affinity capture, dialysis or tangential flow filtration.

In another aspect of the present invention, the anti-CD37 antibody is linked to cytotoxic drugs via disulfide bonds and a polyethylene glycol spacer in enhancing the potency, solubility or the efficacy of the immunoconjugate. Such cleavable hydrophilic linkers are described in WO2009/134977. The additional benefit of this linker design is the desired high monomer ratio and the minimal aggregation of the antibody-drug conjugate. Specifically contemplated in this aspect are conjugates of cell-binding agents and drugs linked via disulfide group (—S—S—) bearing polyethylene glycol spacers ((CH₂CH₂O)ₙ₌₁₋₁₄) with a narrow range of drug load of 2-8 are described that show relatively high potent biological activity toward cancer cells and have the desired biochemical properties of high conjugation yield and high monomer ratio with minimal protein aggregation.

Specifically contemplated in this aspect is an anti-CD37 antibody drug conjugate of formula (I) or a conjugate of formula (I′):

CB—[X₁—(—CH₂—CH₂O—)ₙ—Y—D]ₘ (I)

[D-Y—(—CH₂—CH₂O—)ₙ—X₁]ₘ—CB (I′)

wherein:

CB represents an anti-CD37 antibody or fragment;

D represents a drug;

X represents an aliphatic, an aromatic or a heterocyclic unit attached to the cell-binding agent via a thioether bond, an amide bond, a carbamate bond, or an ether bond;

Y represents an aliphatic, an aromatic or a heterocyclic unit attached to the drug via a disulfide bond;

1 is 0 or 1;

m is an integer from 2 to 8; and

n is an integer from 1 to 24.

In some embodiments, m is an integer from 2 to 6.

In some embodiments, m is an integer from 3 to 5.

In some embodiments, n is an integer form 2 to 8. Alternatively, as disclosed in, for example, U.S. Pat. Nos. 6,441,163 and 7,368,565, the drug can be first modified to introduce a reactive ester suitable to react with a cell-binding agent. Reaction of these drugs containing an activated linker moiety with a cell-binding agent provides another method of producing a cell-binding agent drug conjugate. Maytansinoids can also be linked to an anti-CD37 antibody or fragment using PEG linking groups, as set forth for example in U.S. Pat. No. 6,716,821. These PEG non-cleavable linking groups are soluble both in water and in non-aqueous solvents, and can be used to join one or more cytotoxic agents to a cell binding agent. Exemplary PEG linking groups include heterobifunctional PEG linkers that react with cytotoxic agents and cell binding agents at opposite ends of the linkers through a functional sulfhydryl or disulfide group at one end, and an active ester at the other end. As a general example of the synthesis of a cytotoxic conjugate using a PEG linking group, reference is again made to U.S. Pat. No. 6,716,821 which is incorporated entirely by reference herein. Synthesis begins with the reaction of one or more cytotoxic agents bearing a reactive PEG moiety with a cell-binding agent, resulting in displacement of the terminal active ester of each reactive PEG moiety by an amino acid residue of the cell binding agent, to yield a cytotoxic conjugate comprising one or more cytotoxic agents covalently bonded to a cell binding agent through a PEG linking group. Alternatively, the cell binding can be modified with the bifunctional PEG crosslinker to introduce a reactive disulfide moiety (such as a pyridyldisulfide), which can then be treated with a thiol-containing maytansinoid to provide a conjugate. In another method, the cell binding can be modified with the bifunctional PEG crosslinker to introduce a thiol moiety which can then can be treated with a reactive disulfide-containing maytansinoid (such as a pyridyldisulfide), to provide a conjugate.

Antibody-maytansinoid conjugates with non-cleavable linkers can also be prepared. Such crosslinkers are described in the art (see US Publication No. 2005/0169933) and include but are not limited to, N-succinimidyl 4-(maleimidomethyl) cyclohexanecarboxylate (SMCC). In some embodiments, the antibody is modified with crosslinking reagents such as succinimidyl 4-(N-maleimidomethyl)-cyclohexane-1-carboxylate (SMCC), sulfo-SMCC, maleimidobenzoyl-N-hydroxysuccinimide ester (MBS), sulfo-MBS or succinimidyl-iodoacetate, as described in the literature, to introduce 1-10 reactive groups (Yoshitake et al, Eur. J. Biochem., 101:395-399 (1979); Hashida et al, J. Applied Biochem., 56-63 (1984); and Liu et al, Biochem., 18:690-697 (1979)). The modified antibody is then reacted with the thiol-containing maytansinoid derivative to produce a conjugate. The conjugate can be purified by gel filtration through a Sephadex G25 column or by dialysis or tangential flow filtration. The modified antibodies are treated with the thiol-containing maytansinoid (1 to 2 molar equivalent/maleimido group) and antibody-maytansinoid conjugates are purified by gel filtration through a Sephadex G-25 column, chromatography on a ceramic hydroxyapatite column, dialysis or tangential flow filtration or a combination of methods thereof. Typically, an average of 1-10 maytansinoids per antibody are linked. One method is to modify antibodies with succinimidyl 4-(N-maleimidomethyl)-cyclohexane-1-carboxylate (SMCC) to introduce maleimido groups followed by reaction of the modified antibody with a thiol-containing maytansinoid to give a thioether-linked conjugate. Again conjugates with 1 to 10 drug molecules per antibody molecule result. Maytansinoid conjugates of antibodies, antibody fragments, and other proteins are made in the same way.

In another aspect of the invention, the CD37 antibody is linked to the drug via a non-cleavable bond through the intermediacy of a PEG spacer. Suitable crosslinking reagents comprising hydrophilic PEG chains that form linkers between a drug and the anti-CD37 antibody or fragment are also well known in the art, or are commercially available (for example from Quanta Biodesign, Powell, Ohio). Suitable PEG-containing crosslinkers can also be synthesized from commercially available PEGs themselves using standard synthetic chemistry techniques known to one skilled in the art. The drugs can be reacted with bifunctional PEG-containing cross linkers to give compounds of the following formula, Z—X₁—(—CH₂—CH₂—O—)ₙ—Yₚ-D, by methods described in detail in US Patent Publication 2009/0274713 and in WO2009/134977, which can then react with the cell binding agent to provide a conjugate. Alternatively, the cell binding can be modified with the bifunctional PEG crosslinker to introduce a thiol-reactive group (such as a maleimide or haloacetamide) which can then be treated with a thiol-containing maytansinoid to provide a conjugate. In another method, the cell binding can be modified with the bifunctional PEG crosslinker to introduce a thiol moiety which can then be treated with a thiol-reactive maytansinoid (such as a maytansinoid bearing a maleimide or haloacetamide), to provide a conjugate.

Accordingly, another aspect of the present invention is an anti-CD37 antibody drug conjugate of formula (II) or of formula (II′):

CB—[X₁—(—CH₂—CH₂—O—)ₙ—Yₚ-D]ₘ (II)

[D-Yₚ—(—CH₂—CH₂—O—)ₙ—X₁]ₘ—CB (II′)

wherein, CB represents an anti-CD37 antibody or fragment;

D represents a drug;

X represents an aliphatic, an aromatic or a heterocyclic unit bonded to the cell-binding agent via a thioether bond, an amide bond, a carbamate bond, or an ether bond;

Y represents an aliphatic, an aromatic, or a heterocyclic unit bonded to the drug via a covalent bond selected from the group consisting of a thioether bond, an amide bond, a carbamate bond, an ether bond, an amine bond, a carbon-carbon bond and a hydrazone bond;

1 is 0 or 1;

p is 0 or 1;

m is an integer from 2 to 15; and

n is an integer from 1 to 2000.

In some embodiments, m is an integer from 2 to 8; and

In some embodiments, n is an integer from 1 to 24.

In some embodiments, m is an integer from 2 to 6.

In some embodiments, m is an integer from 3 to 5.

In some embodiments, n is an integer from 2 to 8. Examples of suitable PEG-containing linkers include linkers having an N-succinimidyl ester or N-sulfosuccinimidyl ester moiety for reaction with the anti-CD37 antibody or fragment thereof, as well as a maleimido- or haloacetyl-based moiety for reaction with the compound. A PEG spacer can be incorporated into any crosslinker known in the art by the methods described herein.

In some embodiments, the linker is a linker containing at least one charged group as described, for example, in U.S. Patent Publication No. 2012/0282282, the contents of which are entirely incorporated herein by reference. In some embodiments, the charged or pro-charged cross-linkers are those containing sulfonate, phosphate, carboxyl or quaternary amine substituents that significantly increase the solubility of the modified cell-binding agent and the cell-binding agent-drug conjugates, especially for monoclonal antibody-drug conjugates with 2 to 20 drugs/antibody linked. Conjugates prepared from linkers containing a pro-charged moiety would produce one or more charged moieties after the conjugate is metabolized in a cell. In some embodiments, the linker is selected from the group consisting of: N-succinimidyl 4-(2-pyridyldithio)-2-sulfopentanoate (sulfo-SPP) and N-succinimidyl 4-(2-pyridyldithio)-2-sulfobutanoate (sulfo-SPDB).

Many of the linkers disclosed herein are described in detail in U.S. Patent Publication Nos. 2005/0169933, 2009/0274713, and 2012/0282282, and in WO2009/134977; the contents of which are entirely incorporated herein by reference.

The present invention includes aspects wherein about 2 to about 8 drug molecules (“drug load”), for example, maytansinoid, are linked to an anti-CD37 antibody or fragment thereof, the anti-tumor effect of the conjugate is much more efficacious as compared to a drug load of a lesser or higher number of drugs linked to the same cell binding agent. “Drug load”, as used herein, refers to the number of drug molecules (e.g., a maytansinoid) that can be attached to a cell binding agent (e.g., an anti-CD37 antibody or fragment thereof). In one aspect the number of drug molecules that can be attached to a cell binding agent can average from about 2 to about 8 (e.g., 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1). N^{2′}-deacetyl-N^{2′}-(3-mercapto-1-oxopropyl)-maytansine (DM1) can be used.

The anti-CD37 antibody or fragment thereof can be modified by reacting a bifunctional crosslinking reagent with the anti-CD37 antibody or fragment thereof, thereby resulting in the covalent attachment of a linker molecule to the anti-CD37 antibody or fragment thereof. As used herein, a “bifunctional crosslinking reagent” is any chemical moiety that covalently links a cell-binding agent to a drug, such as the drugs described herein. In another method, a portion of the linking moiety is provided by the drug. In this respect, the drug comprises a linking moiety that is part of a larger linker molecule that is used to join the cell-binding agent to the drug. For example, to form the maytansinoid DM1, the side chain at the C-3 hydroxyl group of maytansine is modified to have a free sulfhydryl group (SH). This thiolated form of maytansine can react with a modified cell-binding agent to form a conjugate. Therefore, the final linker is assembled from two components, one of which is provided by the crosslinking reagent, while the other is provided by the side chain from DM1.

Thus, in one aspect, an immunoconjugate comprises 1 maytansinoid per antibody. In another aspect, an immunoconjugate comprises 2 maytansinoids per antibody. In another aspect, an immunoconjugate comprises 3 maytansinoids per antibody. In another aspect, an immunoconjugate comprises 4 maytansinoids per antibody. In another aspect, an immunoconjugate comprises 5 maytansinoids per antibody. In another aspect, an immunoconjugate comprises 6 maytansinoids per antibody. In another aspect, an immunoconjugate comprises 7 maytansinoids per antibody. In another aspect, an immunoconjugate comprises 8 maytansinoids per antibody.

In one aspect, an immunoconjugate comprises about 1 to about 8 maytansinoids per antibody. In another aspect, an immunoconjugate comprises about 2 to about 7 maytansinoids per antibody. In another aspect, an immunoconjugate comprises about 2 to about 6 maytansinoids per antibody. In another aspect, an immunoconjugate comprises about 2 to about 5 maytansinoids per antibody. In another aspect, an immunoconjugate comprises about 3 to about 5 maytansinoids per antibody. In another aspect, an immunoconjugate comprises about 3 to about 4 maytansinoids per antibody.

In one aspect, a composition comprising immunoconjugates has an average of about 2 to about 8 (e.g., 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1) drug molecules (e.g., maytansinoids) attached per antibody. In one aspect, a composition comprising immunoconjugates has an average of about 1 to about 8 drug molecules (e.g., maytansinoids) per antibody. In one aspect, a composition comprising immunoconjugates has an average of about 2 to about 7 drug molecules (e.g., maytansinoids) per antibody. In one aspect, a composition comprising immunoconjugates has an average of about 2 to about 6 drug molecules (e.g., maytansinoids) per antibody. In one aspect, a composition comprising immunoconjugates has an average of about 2 to about 5 drug molecules (e.g., maytansinoids) per antibody. In one aspect, a composition comprising immunoconjugates has an average of about 3 to about 5 drug molecules (e.g., maytansinoids) per antibody. In one aspect, a composition comprising immunoconjugates has an average of about 3 to about 4 drug molecules (e.g., maytansinoids) per antibody.

In one aspect, a composition comprising immunoconjugates has an average of about 2±0.5, about 3±0.5, about 4±0.5, about 5±0.5, about 6±0.5, about 7±0.5, or about 8±0.5 drug molecules (e.g., maytansinoids) attached per antibody. In one aspect, a composition comprising immunoconjugates has an average of about 3.5±0.5 drug molecules (e.g., maytansinoids) per antibody.

The drug molecules can also be linked to the antibody molecules through an intermediary carrier molecule such as serum albumin.

As used herein, the expression “linked to a cell-binding agent” or “linked to an anti-CD37 antibody or fragment” refers to the conjugate molecule comprising at least one drug derivative bound to a cell-binding agent anti-CD37 antibody or fragment via a suitable linking group, or a precursor thereof. One linking group is SMCC.

In certain embodiments, cytotoxic agents useful in the present invention are maytansinoids and maytansinoid analogs. Examples of suitable maytansinoids include esters of maytansinol and maytansinol analogs. Included are any drugs that inhibit microtubule formation and that are highly toxic to mammalian cells, as are maytansinol and maytansinol analogs.

Examples of suitable maytansinol esters include those having a modified aromatic ring and those having modifications at other positions. Such suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 5,208,020; 5,416,064; 5,475,092; 5,585,499; 5,846,545; 6,333,410; 7,276,497 and 7,473,796.

In a certain embodiment, the immunoconjugates of the invention utilize the thiol-containing maytansinoid (DM1), formally termed N^{2′}-deacetyl-N^{2′}-(3-mercapto-1-oxopropyl)-maytansine, as the cytotoxic agent. DM1 is represented by the following structural formula (I):

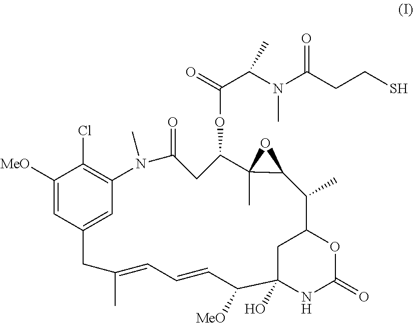 [SMILES: COc1cc2cc(c1Cl)N(C)C(=O)C[C@H](OC(=O)[C@H](C)N(C)C(=O)CCS)[C@]1(C)O[C@H]1[C@H](C)C1C[C@@](O)(NC(=O)O1)[C@H](OC)/C=C/C=C(\C)C2]

In another embodiment, the conjugates of the present invention utilize the thiol-containing maytansinoid N^{2′}-deacetyl-N^{2′}(4-methyl-4-mercapto-1-oxopentyl)-maytansine (e.g., DM4) as the cytotoxic agent. DM4 is represented by the following structural formula (II):

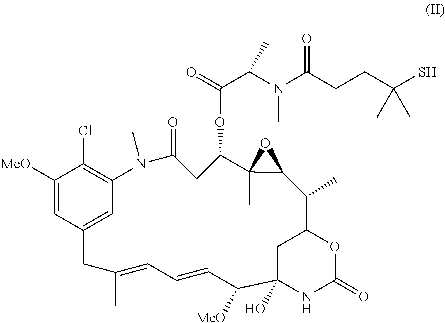 [SMILES: COc1cc2cc(c1Cl)N(C)C(=O)C[C@H](OC(=O)[C@H](C)N(C)C(=O)CCC(C)(C)S)[C@]1(C)O[C@H]1[C@H](C)C1C[C@@](O)(NC(=O)O1)[C@H](OC)/C=C/C=C(\C)C2]

Another maytansinoid comprising a side chain that contains a sterically hindered thiol bond is N^{2′}-deacetyl-N-^{2′}(4-mercapto-1-oxopentyl)-maytansine (termed DM3), represented by the following structural formula (III):

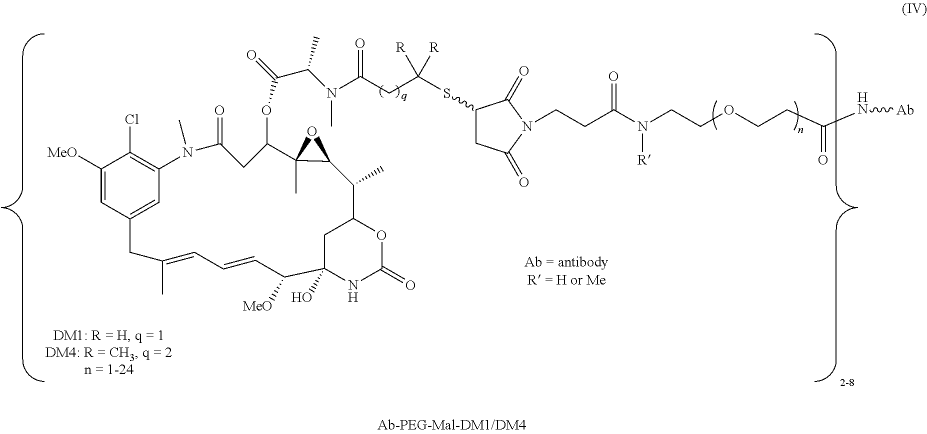

Each of the maytansinoids taught in U.S. Pat. Nos. 5,208,020 and 7,276,497, can also be used in the conjugate of the present invention. In this regard, the entire disclosure of 5,208,020 and 7,276,697 is incorporated herein by reference.

Many positions on maytansinoids can serve as the position to chemically link the linking moiety. For example, the C-3 position having a hydroxyl group, the C-14 position modified with hydroxymethyl, the C-15 position modified with hydroxy and the C-20 position having a hydroxy group are all expected to be useful. In some embodiments, the C-3 position serves as the position to chemically link the linking moiety, and in some particular embodiments, the C-3 position of maytansinol serves as the position to chemically link the linking moiety.

Structural representations of some conjugates are shown below:

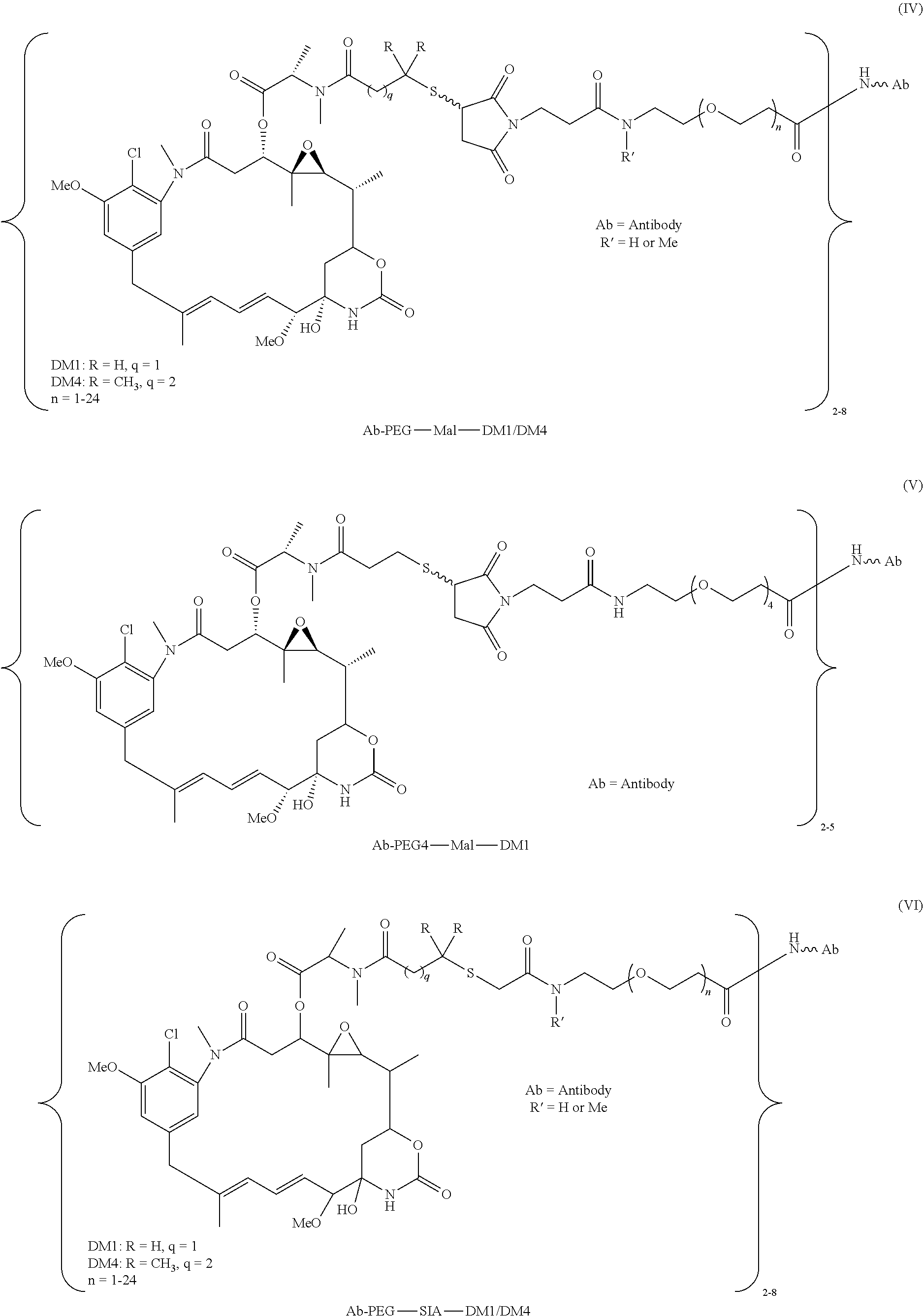

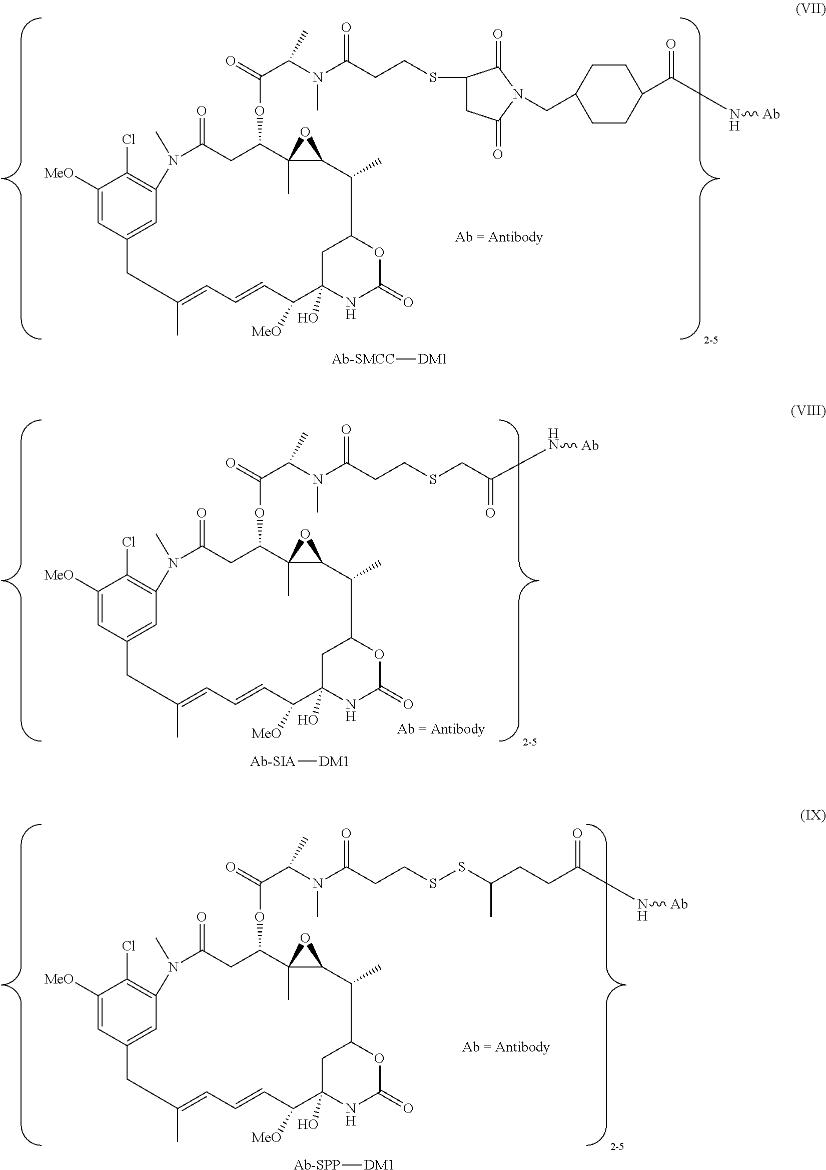

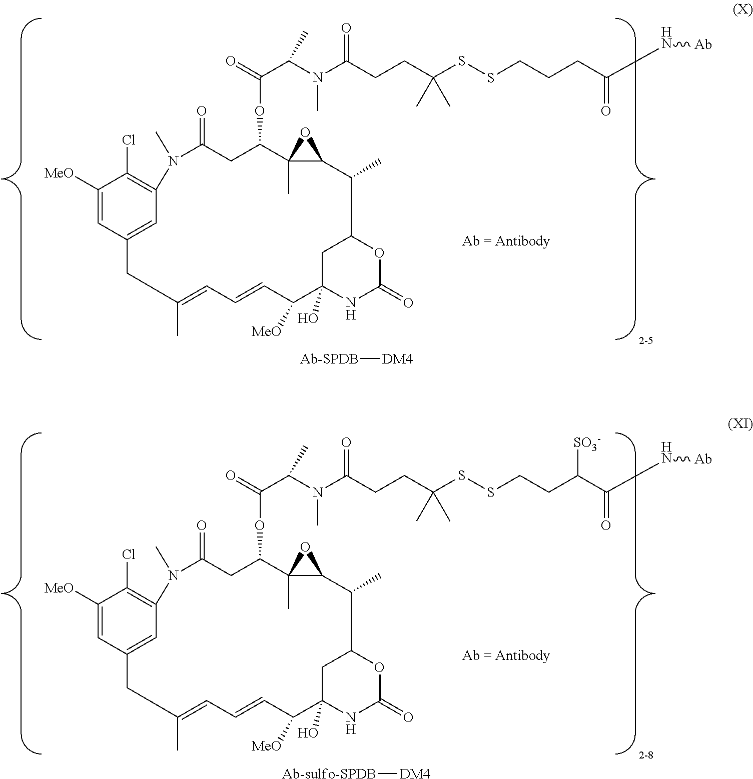

Also included in the present invention are any stereoisomers and mixtures thereof for any compounds or conjugates depicted by any structures above.

Several descriptions for producing such antibody-maytansinoid conjugates are provided in U.S. Pat. Nos. 6,333,410, 6,441,163, 6,716,821, and 7,368,565, each of which is incorporated herein in its entirety.

In general, a solution of an antibody in aqueous buffer can be incubated with a molar excess of maytansinoids having a disulfide moiety that bears a reactive group. The reaction mixture can be quenched by addition of excess amine (such as ethanolamine, taurine, etc.). The maytansinoid-antibody conjugate can then be purified by gel filtration.

The number of maytansinoid molecules bound per antibody molecule can be determined by measuring spectrophotometrically the ratio of the absorbance at 252 nm and 280 nm. The average number of maytansinoid molecules/antibody can be, for example, 1-10 or 2-5. The average number of maytansinoid molecules/antibody can be, for example about 3 to about 4. The average number of maytansinoid molecules/antibody can be about 3.5.

Conjugates of antibodies with maytansinoid or other drugs can be evaluated for their ability to suppress proliferation of various unwanted cell lines in vitro. For example, cell lines such as the human lymphoma cell line Daudi and the human lymphoma cell line Ramos, can easily be used for the assessment of cytotoxicity of these compounds. Cells to be evaluated can be exposed to the compounds for 4 to 5 days and the surviving fractions of cells measured in direct assays by known methods. IC₅₀ values can then be calculated from the results of the assays.

The immunoconjugates can, according to some embodiments described herein, be internalized into cells. The immunoconjugate, therefore, can exert a therapeutic effect when it is taken up by, or internalized, by a CD37-expressing cell. In some particular embodiments, the immunoconjugate comprises an antibody, antibody fragment, or polypeptide, linked to a cytotoxic agent by a cleavable linker, and the cytotoxic agent is cleaved from the antibody, antibody fragment, or polypeptide, wherein it is internalized by a CD37-expressing cell.

### IV. METHODS OF USE AND PHARMACEUTICAL COMPOSITIONS

The CD37-binding agents (including antibodies, immunoconjugates, and polypeptides) of the invention are useful in a variety of applications including, but not limited to, therapeutic treatment methods, such as the treatment of cancer, such as B-cell malignancies. In certain embodiments, the agents are useful for inhibiting tumor growth, inducing differentiation, reducing tumor volume, and/or reducing the tumorigenicity of a tumor. The methods of use can be in vivo methods.

In certain embodiments, the dosage of the immunoconjugate is from about 0.1 to 3.0 mg of the CD37-binding agent per kg of body weight (mg/kg). In certain embodiments, the dosage of the immunoconjugate is from 0.4 to 0.8 mg per kg of body weight. In certain embodiments, the dosage of the immunoconjugate is from 0.8 to 1.4 mg per kg of body weight. In certain embodiments, the dosage of the immunoconjugate is from 0.8 to 1.2 mg per kg of body weight. In certain embodiments, the dosage of the immunoconjugate is from 1.0 to 3.0 mg per kg of body weight. In certain embodiments, the dosage of the immunoconjugate is from 1.0 to 2.8 mg per kg of body weight. In certain embodiments, the dosage of the immunoconjugate is from about 1.0 to about 1.4 mg per kg of body weight. In certain embodiments, the dosage of the immunoconjugate is from about 1.4 to about 2.0 mg per kg of body weight. In certain embodiments, the dosage of immunoconjugate is from about 1.4 to about 3.0 mg per kg of body weight. In certain embodiments, the dosage of the immunoconjugate is from about 1.4 to about 2.8 mg per kg of body weight. In certain embodiments, the dosage of the immunoconjugate is from about 2.0 to about 2.8 mg per kg of body weight. In certain embodiments, the dosage of the immunoconjugate is from about 2.0 to about 3.0 mg per kg of body weight. In certain embodiments, the dosage of immunoconjugate is about 0.1 per kg of body weight. In certain embodiments, the immunoconjugate is about 0.2 mg per kg of body weight. In certain embodiments, the dosage of the immunoconjugate is about 0.3 mg per kg of body weight. In certain embodiments, the dosage of immunoconjugate is about 0.4 mg per kg of body weight. In certain embodiments, the dosage of the immunoconjugate is about 0.5 mg per kg of body weight. In certain embodiments, the dosage of the immunoconjugate is about 0.6 mg per kg of body weight. In certain embodiments, the dosage of the immunoconjugate is about 0.7 mg per kg of body weight. In certain embodiments, the dosage of the immunoconjugate is about 0.8 mg per kg of body weight. In certain embodiments, the dosage of the immunoconjugate is about 0.9 mg per kg of body weight. In certain embodiments, the dosage of the immunoconjugate is about 1.0 mg per kg of body weight. In certain embodiments, the dosage of the immunoconjugate is about 1.1 mg per kg of body weight. In certain embodiments, the dosage of the immunoconjugate is about 1.2 mg per kg of body weight. In certain embodiments, the dosage of the immunoconjugate is about 1.3 mg per kg of body weight. In certain embodiments, the dosage of the immunoconjugate is about 1.4 mg per kg of body weight. In certain embodiments, the dosage of the immunoconjugate is about 1.5 mg per kg of body weight. In certain embodiments, the dosage of the immunoconjugate is about 1.6 mg per kg of body weight. In certain embodiments, the dosage of the immunoconjugate is about 1.7 mg per kg of body weight. In certain embodiments, the dosage of the immunoconjugate is about 1.8 mg per kg of body weight. In certain embodiments, the dosage of the immunoconjugate is about 1.9 mg per kg of body weight. In certain embodiments, the dosage of the immunoconjugate is about 2.0 mg per kg of body weight. In certain embodiments, the dosage of immunoconjugate is about 2.1 mg per kg of body weight. In certain embodiments, the dosage of the immunoconjugate is about 2.2 mg per kg of body weight. In certain embodiments, the dosage of the immunoconjugate is about 2.3 mg per kg of body weight. In certain embodiments, the dosage of the immunoconjugate is about 2.4 mg per kg of body weight. In certain embodiments, the dosage of the immunoconjugate is about 2.5 mg per kg of body weight. In certain embodiments, the dosage of the immunoconjugate is about 2.6 mg per kg of body weight. In certain embodiments, the dosage of the immunoconjugate is about 2.7 mg per kg of body weight. In certain embodiments, the dosage of the immunoconjugate is about 2.8 mg per kg of body weight. In certain embodiments, the dosage of the immunoconjugate is about 2.9 mg per kg of body weight. In certain embodiments, the dosage of the immunoconjugate is about 3.0 mg per kg of body weight.

In certain embodiments, the disease treated with the CD37-binding agent or antagonist (e.g., an anti-CD37 antibody) is a cancer. In certain embodiments, the cancer is characterized by CD37 expressing cells to which the CD37-binding agent (e.g., antibody) binds.

The present invention provides for methods of treating cancer comprising administering a therapeutically effective amount of a CD37-binding agent to a subject (e.g., a subject in need of treatment). In certain embodiments, the cancer is a B-cell malignancy. In certain embodiments, the cancer is leukemia or lymphoma. In certain embodiments, the cancer is selected from the group consisting of B cell lymphomas, NHL, precursor B cell lymphoblastic leukemia/lymphoma and mature B cell neoplasms, B cell chronic lymphocytic leukemia (CLL)/small lymphocytic lymphoma (SLL), small cell lymphoma, B cell prolymphocytic leukemia, lymphoplasmacytic lymphoma, mantle cell lymphoma (MCL), follicular lymphoma (FL), low grade, intermediate-grade and high-grade (FL), cutaneous follicle center lymphoma, marginal zone B cell lymphoma, MALT type marginal zone B cell lymphoma, nodal marginal zone B cell lymphoma, splenic type marginal zone B cell lymphoma, hairy cell leukemia, diffuse large B cell lymphoma, Burkitt's lymphoma, plasmacytoma, plasma cell myeloma, post-transplant lymphoproliferative disorder, Waldenstrom's macroglobulinemia, and anaplastic large-cell lymphoma (ALCL). In certain embodiments, the cancer is selected from the group consisting of diffuse large B cell lymphoma (DLBCL), follicular lymphoma (FL), unspecified NHL, MALT lymphoma, mantle cell lymphoma (MCL), Burkitt's lymphoma (BL), and chronic lymphocytic leukemia (CLL). In certain embodiments, the cancer is relapsed or refractory NHL. In certain embodiments, the subject is a human.

In certain embodiments, the method of inhibiting tumor growth comprises administering to a subject a therapeutically effective amount of a CD37-binding agent. In certain embodiments, the subject is a human. In certain embodiments, the subject has a tumor or has had a tumor removed. In certain embodiments, the subject has already received treatment with an anti-CD20 therapy. In certain embodiments, the anti-CD20 therapy includes treatment with an anti-CD20 antibody. In certain embodiments, the anti-CD20 antibody is Rituximab.

In addition, the invention provides a method of reducing the tumorigenicity of a tumor in a subject, comprising administering a therapeutically effective amount of a CD37-binding agent to the subject. In certain embodiments, the tumor comprises cancer stem cells. In certain embodiments, the frequency of cancer stem cells in the tumor is reduced by administration of the agent.

The present invention further provides pharmaceutical compositions comprising one or more of the CD37-binding agents described herein. In certain embodiments, the pharmaceutical compositions further comprise a pharmaceutically acceptable vehicle. These pharmaceutical compositions find use in inhibiting tumor growth and treating cancer in human patients.

In certain embodiments, formulations are prepared for storage and use by combining a purified antibody or agent of the present invention with a pharmaceutically acceptable vehicle (e.g. carrier, excipient) (Remington, The Science and Practice of Pharmacy 20th Edition Mack Publishing, 2000). Suitable pharmaceutically acceptable vehicles include, but are not limited to, nontoxic buffers such as phosphate, citrate, succinate and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g. less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and non-ionic surfactants such as TWEEN or polyethylene glycol (PEG).

The pharmaceutical compositions for use as provided herein can be administered in any number of ways for either local or systemic treatment. Administration can be topical (such as to mucous membranes including vaginal and rectal delivery) such as transdermal patches, ointments, lotions, creams, gels, drops, suppositories, sprays, liquids and powders; pulmonary (e.g., by inhalation or insufflation of powders or aerosols, including by nebulizer; intratracheal, intranasal, epidermal and transdermal); oral; or parenteral including intravenous, intraarterial, subcutaneous, intraperitoneal or intramuscular injection or infusion; or intracranial (e.g., intrathecal or intraventricular) administration. In some embodiments, the administration is intravenous.

An antibody or immunoconjugate of the invention can be combined in a pharmaceutical combination formulation, or dosing regimen as combination therapy, with a second compound having anti-cancer properties. The second compound of the pharmaceutical combination formulation or dosing regimen can have complementary activities to the ADC of the combination such that they do not adversely affect each other. Pharmaceutical compositions comprising the CD37-binding agent and the second anti-cancer agent are also provided. For example, CD37-binding agents can be administered in combination with CD20 antagonists, such as Rituximab. In some embodiments, the subject has already received treatment with an anti-CD20 therapy (e.g., anti-CD20 antibodies including Rituximab). In some embodiments, the antibody or immunoconjugate of the invention can be combined in a pharmaceutical combination formulation, or dosing regimen as combination therapy, with a third, fourth, or additional compounds having anti-cancer properties.

In some embodiments, CD37-binding agents can be administered in combination with cyclophosphamide. In some embodiments, CD37-binding agents can be administered in combination with hydroxydaunorubicin (doxorubicin). In some embodiments, CD37-binding agents can be administered in combination with oncovin (vincristine). In some embodiments, CD37-binding agents can be administered in combination with prednisone or prednisolone. In some embodiments, CD37-binding agents can be administered in combination with cyclophosphamide, hydroxydaunorubicin (doxorubicin), oncovin (vincristine), and prednisone or prednisolone (CHOP). In some embodiments, CHOP is administered in a 3-week (21-day) cycle. In some embodiments, cyclophosphamide, doxorubicin, and vincristine are administered on day 1 and prednisone or prednisolone is administered on days 1-5 of a 3-week (21-day) cycle. In some embodiments, CD37-binding agents can be administered in combination with rituximab, cyclophosphamide, hydroxydaunorubicin (doxorubicin), oncovin (vincristine), and prednisone or prednisolone (R-CHOP).

In some embodiments, the methods further comprise administering a corticosteroid to the patient. In some embodiments the corticosteroid can be selected from the group consisting of prednisone, prednisolone, methylprednisolone, beclamethasone, betamethasone, dexamethasone, fludrocortisone, hydrocortisone, and triamcinolone. In some embodiments, the corticosteroid can be dexamethasone. In some embodiments, the corticosteroid can be administered as a pre-treatment, i.e., prior to the administration of the anti-CD37 binding agent. In some embodiments, the corticosteroid can be administered during the administration of the anti-CD37 binding agent. In some embodiments, the corticosteroid can be administered during the administration of the anti-CD37 binding agent and at least one additional time from about one day after to about five days after the administration of the anti-CD37 binding agent. In some embodiments, the corticosteroid can be administered during the administration of the anti-CD37 binding agent and at least one additional time from about one day after to about four days after the administration of the anti-CD37 binding agent. In some embodiments, the corticosteroid can be administered during the administration of the anti-CD37 binding agent and at least one additional time from about one day after to about three days after the administration of the anti-CD37 binding agent. In some embodiments, the corticosteroid can be administered during the administration of the anti-CD37 binding agent and at least one additional time from about one day after to about two days after the administration of the anti-CD37 binding agent. In some embodiments, the corticosteroid can be administered during the administration of the anti-CD37 binding agent and at least one additional time from about two days after to about five days after the administration of the anti-CD37 binding agent. In some embodiments, the corticosteroid can be administered during the administration of the anti-CD37 binding agent and at least one additional time from about two days after to about four days after the administration of the anti-CD37 binding agent. In some embodiments, the corticosteroid can be administered during the administration of the anti-CD37 binding agent and at least one additional time from about two days after to about three days after the administration of the anti-CD37 binding agent. In some embodiments, the corticosteroid can be administered during the administration of the anti-CD37 binding agent and at about two days after and at about three days after the administration of the anti-CD37 binding agent. In some embodiments, the corticosteroid can be administered during the administration of the anti-CD37 binding agent and at about two days after and at about three days after the administration of the anti-CD37 binding agent. In some embodiments, the corticosteroid can be administered by peri-infusion. In some embodiments, the corticosteroid is administered 30 to 60 minutes prior to administration of the anti-CD37 binding agent. In some embodiments, the corticosteroid is administered 30 to 60 minutes prior to administration of the anti-CD37 binding agent and on at least one additional time on days 1 to 3 following administration of the anti-CD37 binding agent. Pre-infusion intravenous steroid administration was found to eliminate cytokine-mediated adverse effects. In some embodiments, the corticosteroid is administered on at least one of days 2 and 3 following infusion. In some embodiments, the corticosteroid is administered by IV 30 to 60 minutes prior to administration of the anti-CD37 binding agent and orally on days 2 and 3 following infusion.

In some embodiments the corticosteroid is administered by IV. In some embodiments the steroid is administered orally.

In some embodiments, the corticosteroid is administered intravenously 30 to 60 minutes prior to the administration of the anti-CD37 immunoconjugate (e.g., IMGN529) and the corticosteroid is administered orally on days 2 and 3 of a 3-week anti-CD37 immunoconjugate administration cycle.

In some embodiments the corticosteroid to be administered can be dexamethasone. In some embodiments the corticosteroid to be administered can be methylprednisolone. In some embodiments the corticosteroid to be administered can be prednisolone.

In some embodiments, from about 5 mg to about 10 mg dexamethasone is administered. In some embodiments, from about 8 mg to about 10 mg dexamethasone is administered. In some embodiments, about 10 mg dexamethasone is administered. In some embodiments, about 8 mg dexamethasone is administered. In some embodiments about 10 mg dexamethasone is administered by IV 30 to 60 minutes prior to administration of the anti-CD37 binding agent. In some embodiments about 10 mg dexamethasone is administered by IV at the time of administration of the anti-CD37 binding agent and again about 1 to about 5 days after administration of the anti-CD37 binding agent. In some embodiments, the corticosteroid is administered by IV 30 to 60 minutes prior to administration of the anti-CD37 binding agent and one dose of 8 mg of dexamethasone is delivered orally on days 2 and 3 following infusion.

In some embodiments, 10 mg dexamethasone is administered intravenously 30 to 60 minutes prior to the administration of the anti-CD37 immunoconjugate (e.g., IMGN529) and 8 mg dexamethasone is administered orally on days 2 and 3 of a 3-week anti-CD37 immunoconjugate administration cycle.

In some embodiments, the methods further comprise administering a growth factor to the patient. Methods of administering white blood cell growth factors are reviewed, for example, in Smith et al., J. Clin. Oncol. 24: 3187-3205 (2006), which is herein incorporated by reference in its entirety. Growth factor treatment may decrease the likelihood of neutropenias. In some embodiments, the growth factor can be granulocyte colony-stimulating factor (G-CSF). In some embodiments the growth factor can be granulocyte-macrophage colony-stimulating factor (GM-CSF). In some embodiments the growth factor can be macrophage colony-stimulating factor (M-CSF). In some embodiments, the growth factor can be filgrastim. In some embodiments, the growth factor can be pegylated, e.g., pegylated G-CSF. In some embodiments, the growth factor can be pegfilgrastim, marketed as Neulasta®.

In some embodiments, the growth factor can be administered as a pre-treatment, i.e., prior to the administration of the anti-CD37 binding agent. In some embodiments, the anti-CD37 binding agent is administered on a 3-week (about 21-day) cycle and the growth factor can be administered at any point during the 3-week (about 21-day) cycle. In some embodiments, the anti-CD37 binding agent is administered on a 3-week (about 21-day) cycle and the growth factor can be administered early to middle cycle of the 3-week (about 21-day) cycle. In some embodiments, the growth factor can be administered on at least one day from day 1 to about day 21 of the 3-week (about 21-day) cycle. In some embodiments, the growth factor can be administered on at least one day from day 1 to about day 20 of the 3-week (about 21-day) cycle. In some embodiments, the growth factor can be administered on at least one day from day 1 to about day 19 of the 3-week (about 21-day) cycle. In some embodiments, the growth factor can be administered on at least one day from day 1 to about day 18 of the 3-week (about 21-day) cycle. In some embodiments, the growth factor can be administered on at least one day from day 1 to about day 17 of the 3-week (about 21-day) cycle. In some embodiments, the growth factor can be administered on at least one day from day 1 to about day 16 of the 3-week (about 21-day) cycle. In some embodiments, the growth factor can be administered on at least one day from day 1 to about day 14 of the 3-week (about 21-day) cycle. In some embodiments, the growth factor can be administered on at least one day from day 1 to about day 12 of the 3-week (about 21-day) cycle. In some embodiments, the growth factor can be administered on at least one day from day about 15 to about day 21 of the 3-week (about 21-day) cycle. In some embodiments, the growth factor can be administered on at least one day from about day 3 to about day 10 of the 3-week (about 21-day) cycle. In some embodiments, the growth factor can be administered at least twice from about day 3 to about day 10 of the 3-week (about 21-day) cycle. In some embodiments, the growth factor can be administered at least three times from about day 3 to about day 10 of the 3-week (about 21-day) cycle. In some embodiments, the growth factor can be administered on at least one day from about day 4 to about day 10 of the 3-week (about 21-day) cycle. In some embodiments, the growth factor can be administered on at least one day from day 5 to day 8 of the 3-week (about 21-day) cycle. In some embodiments, the growth factor can be administered on at least one day selected from day 5, day 6, and day 8 of the 3-week (about 21-day) cycle. In some embodiments, the growth factor can be administered on days 5, 6, and 8 of the 3-week (about 21-day) cycle.

In some embodiments, G-CSF is administered at a dose of about 1 μg/kg body weight to about 15 μg/kg body weight, per day that the growth factor is administered. In some embodiments, G-CSF is administered at a dose of about 5 μg/kg/day. In some embodiments, G-CSF is administered at a dose of about 10 μg/kg/day.

In some embodiments, G-CSF is administered at a dose of about 200 μg to about 600 μg per day. In some embodiments, G-CSF is administered at a dose of about 300 μg to about 500 μg per day. In some embodiments, G-CSF is administered at a dose of about 300 μg to about 480 μg per day. In some embodiments, G-CSF is administered at a dose of about 300 μg/day. In some embodiments, G-CSF is administered at a dose of about 400 μg/day. In some embodiments, G-CSF is administered at a dose of about 480 μg/day. In some embodiments, G-CSF is administered at a dose of about 500 μg/day.

In some embodiments, GM-CSF is administered at a dose of about 100 μg/m² to about 500 μg/m², per day that the growth factor is administered. In some embodiments, GM-CSF is administered at a dose of about 250 μg/m²/day.

In some embodiments, GM-CSF is administered at a dose of about 200 μg to about 600 μg per day. In some embodiments, GM-CSF is administered at a dose of about 300 μg to about 500 μg per day. In some embodiments, GM-CSF is administered at a dose of about 300 μg to about 480 μg per day. In some embodiments, GM-CSF is administered at a dose of about 300 μg/day. In some embodiments, G-CSF is administered at a dose of about 400 μg/day. In some embodiments, GM-CSF is administered at a dose of about 480 μg/day. In some embodiments, GM-CSF is administered at a dose of about 500 μg/day.

In some embodiments, pegfilgrastim is administered at a dose of about 6 mg per cycle. In some embodiments, pegfilgrastim is administered at a dose of about 10 μg/kg to about 500 μg/kg per cycle. In some embodiments, pegfilgrastim is administered at a dose of about 10 μg/kg to about 400 μg/kg per cycle. In some embodiments, pegfilgrastim is administered at a dose of about 50 μg/kg to about 300 μg/kg per cycle. In some embodiments, pegfilgrastim is administered at a dose of about 50 μg/kg to about 200 μg/kg per cycle. In some embodiments, pegfilgrastim is administered at a dose of about 50 μg/kg to about 150 μg/kg per cycle. In some embodiments, pegfilgrastim is administered at a dose of about 100 μg/kg per cycle.

In some embodiments, administration of corticosteroids and/or G-CSF to the dosing protocol allows a higher dose to be administered. In some embodiments, patients stay on the treatment longer due to the administration of corticosteroids and/or G-CSF. In some embodiments, less neutropenia is observed due to the administration of corticosteroids and/or G-CSF. In some embodiments, more clinical benefits are observed due to the administration of corticosteroids and/or G-CSF.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, can be practiced without departing from the scope of the present disclosure.

### EXAMPLES

It is understood that the examples and embodiments described herein are for illustrative purposes only and that various modifications or changes in light thereof will be suggested to persons skilled in the art and are to be included within the spirit and purview of this application

### Example 1

### IMGN529 Dosing Trial in Human Cancer Patients

IMGN529 is a CD37-targeting antibody-drug conjugate (ADC) comprising a CD37-binding antibody conjugated to the maytansinoid anti-mitotic, DM1. IMGN529 is huCD37-3-SMCC-DM1, and the huCD37-3 antibody contains a variable heavy chain with the amino acid sequence of SEQ ID NO:12 and a variable light chain with the amino acid sequence of SEQ ID NO:15.

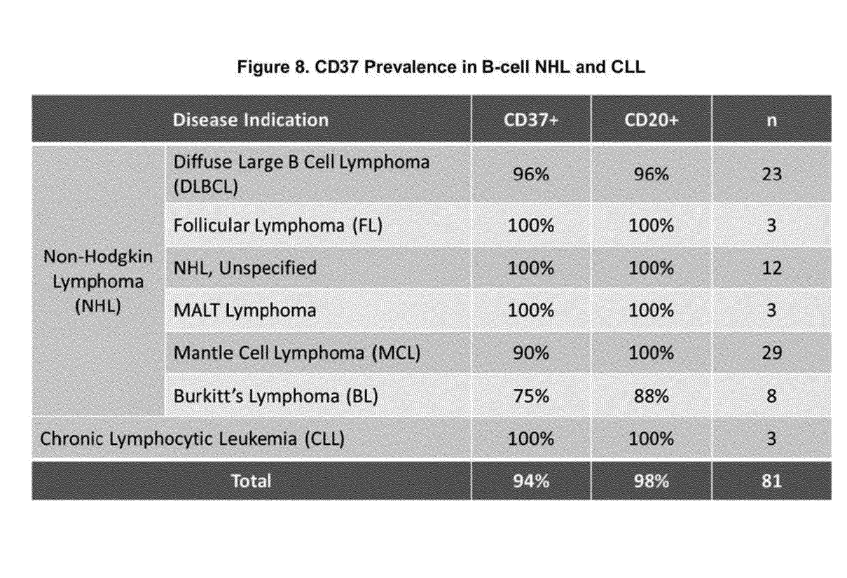

CD37 is expressed on the surface of normal B cells, during the pre-B to peripheral mature B-cell stages, and on malignant B-cells, such as those found in non-Hodgkin's lymphoma (NHL) and chronic lymphoid leukemia (CLL). IHC staining of lymphoma tissue shows that CD37 has similar prevalence in NHL subtypes as CD20 (FIG. 8). In preclinical studies, IMGN529 exhibits potent antitumor activity against NHL cells via direct inhibition, effector function, and delivery of the maytansinoid payload.

A study to determine the maximum tolerated dose (MTD) and recommended phase 2 dose (RP2D) as well as to evaluate the safety, pharmacokinetics (PK), pharmacodynamics (PD), and efficacy of IMGN529 was initiated. The study employed a standard 3+3 design. The MTD is defined as the highest dose at which no more than 1 of 6 patients (<33%) experiences a dose limiting toxicity (DLT) in the dose cohort.

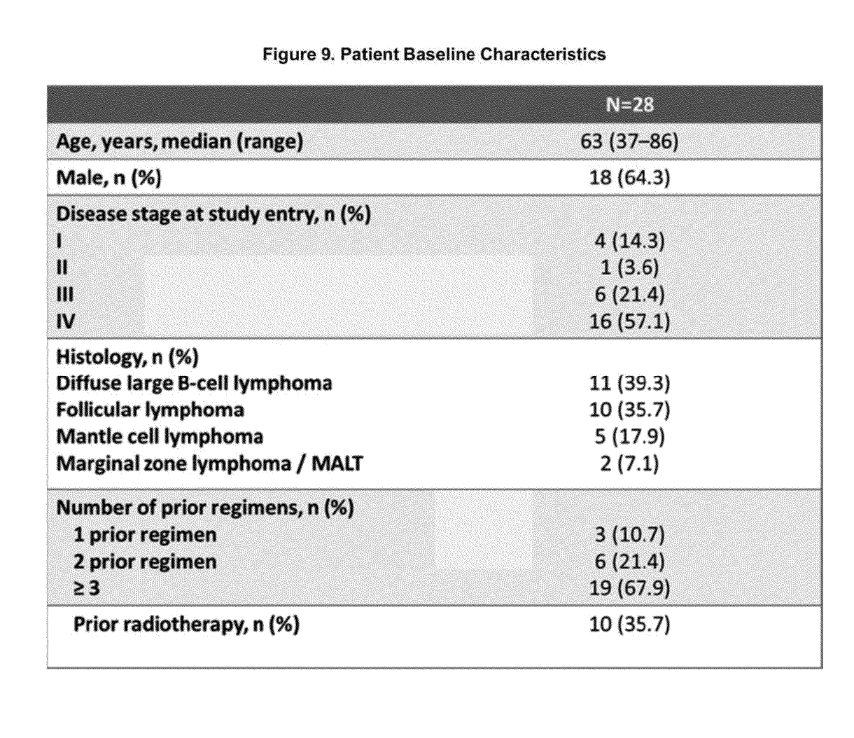

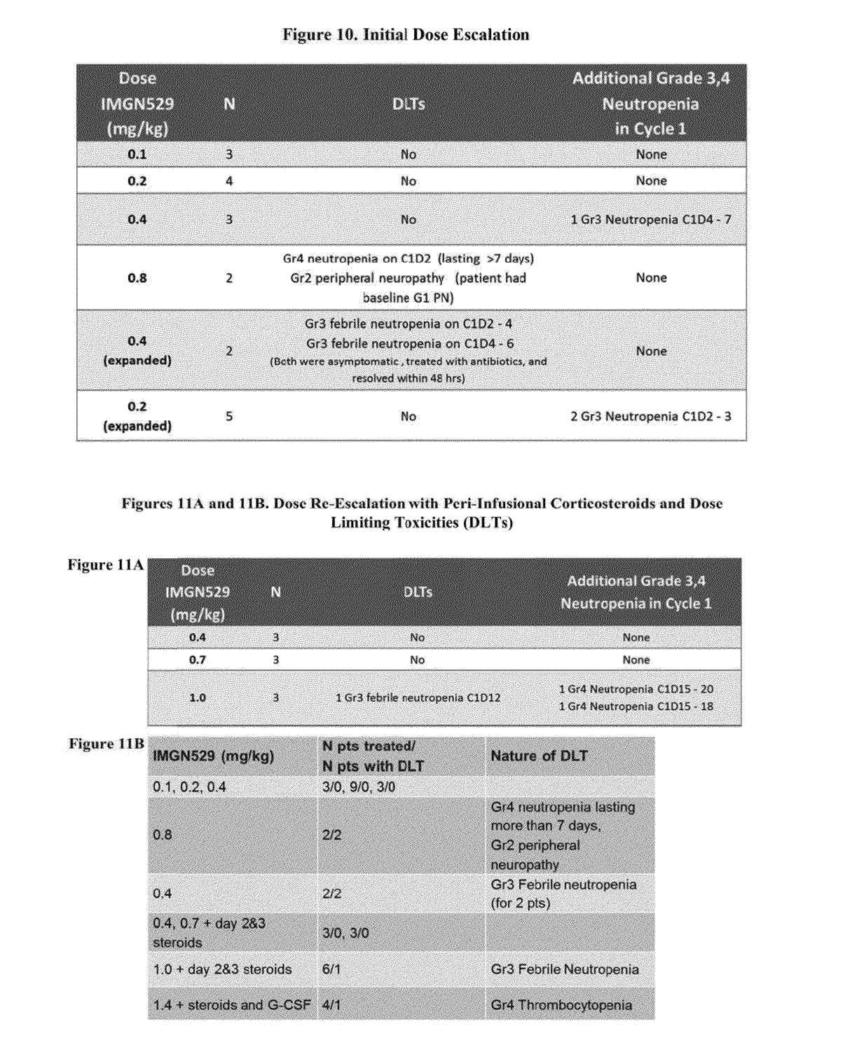

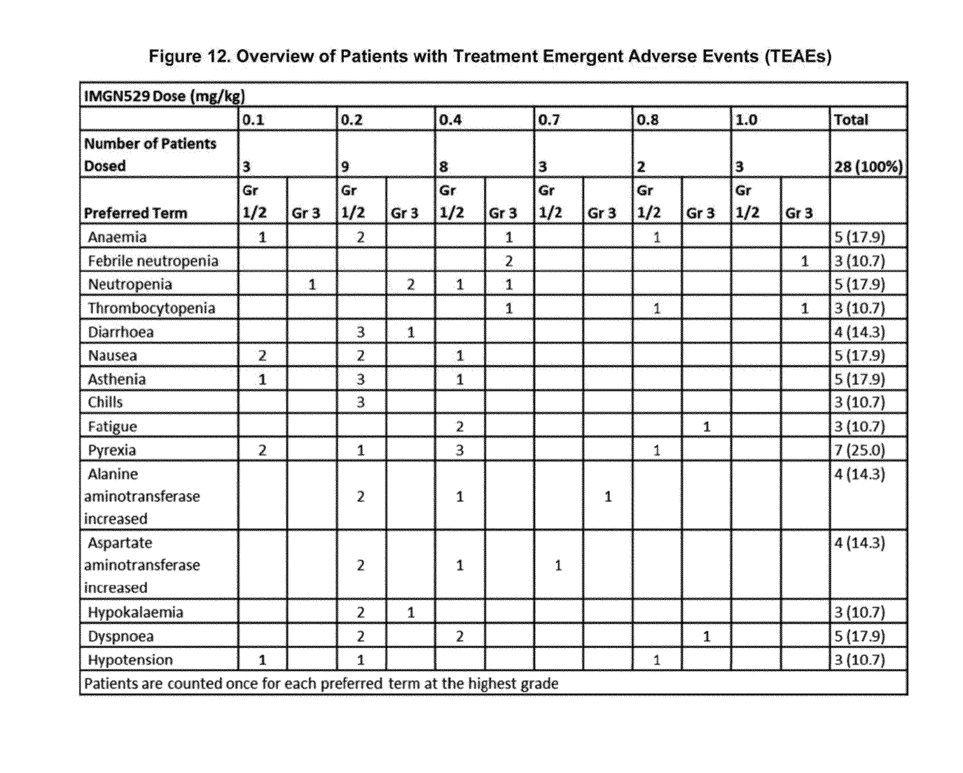

In the study, patients received IMGN529 intravenously (IV) on day 1 of a 21-day (3 week) cycle. Twenty-eight patients were enrolled and their baseline characteristics are shown in FIG. 9. These patients received dose levels ranging from 0.1 to 1.0 mg/kg IMGN529: eleven patients with diffuse large B-cell lymphoma (DLBCL), ten patients with follicular lymphoma (FL), five patients with mantle cell lymphoma, and two patients with marginal zone/MALT. The adverse events (AEs) are shown in FIGS. 10-12. At the 0.8 mg/kg IMGN529 dose, two patients reported dose-limiting toxicities (one patient reported Grade 2 peripheral neuropathy and one patient reported Grade 4 neutropenia lasting longer than seven days). At the 0.4 mg/kg IMGN529 dose level, two patients reported Grade 3 febrile neutropenia. Other events of early onset (day 1 to day 5) Grade 3 neutropenia were also reported in some patients. Additionally, one patient with DLBCL treated at 0.4 mg/kg IMGN529 and one patient with FL treated at 0.2 mg/kg achieved partial remission in C3 and C5 respectively.

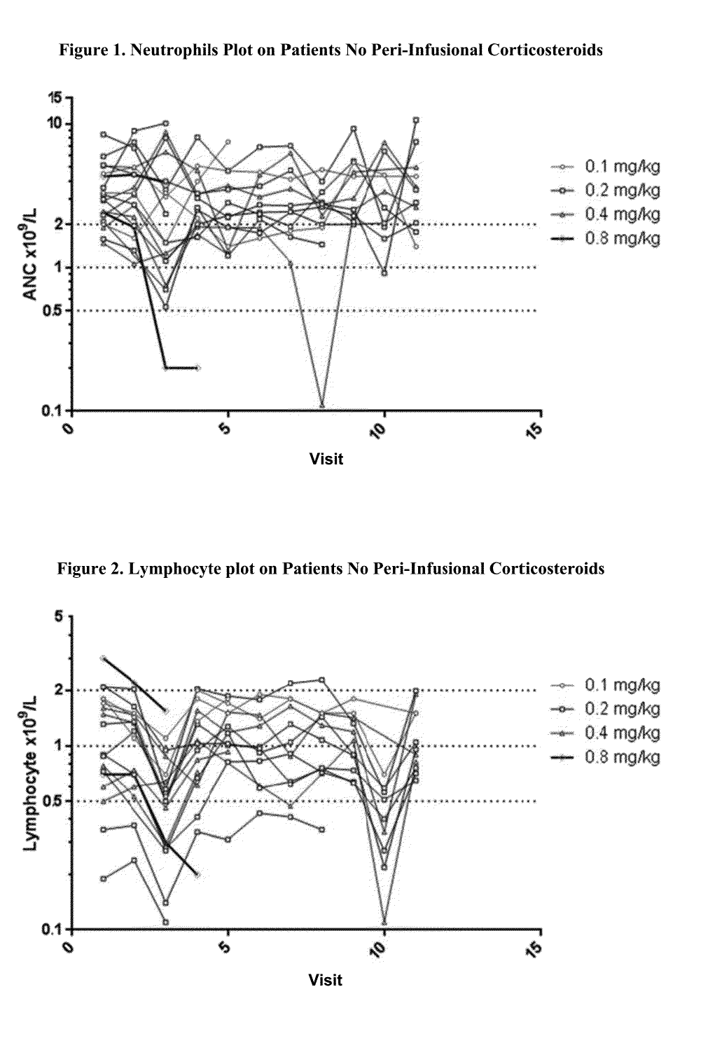

The absolute neutrophil count (ANC) and lymphocyte levels of treated patients are shown in FIGS. 1 and 2, respectively.

Because transient grade 3-4 neutropenia occurred soon after dosing in a subset of patients receiving doses at or below 0.8 mg/kg, additional patients were treated as described in more detail in Examples 2 and 3 below. In short, peri-infusional steroid administration was added to the study protocol, and the incidence and severity of this neutropenia was significantly reduced. At the dose of 1.0 mg/kg with peri-infusional prophylaxis, the first patient had G3 febrile neutropenia at day 12, and the subsequent two patients had G4 neutropenia at day 15. G-CSF support was subsequently added, and no other incidences of febrile neutropenia were reported in additional patients. Overall the incidence of neutropenia and/or febrile neutropenia declined after adding corticosteroids and G-CSF.

IMGN529 showed encouraging anti-tumor activity. Of the ten evaluable relapsed or refractory patients, four achieved a response: one achieved a complete response, and three achieved partial responses. In addition, one Grade 3 FL patient achieved a partial response. In addition to these responses, one patient had a tumor lysis syndrome, and the majority of patients achieved a reduction in lymphocyte count on day 2 after dosing, suggestive of a CD37-mediated reduction in lymphocytes. These observations are consistent with the mechanism of action of a CD37-targeted therapy.

IMGN529 exhibited a manageable toxicity profile with the most common grade 3-4 AEs being hematologic in nature. A summary of DLTs observed is shown in FIG. 11B.

The pharmacokinetics of IMGN529 are non-linear. The exposures of IMGN529 increased with an increase in dose in a manner of greater than dose-proportional. The mean apparent elimination half-life of IMGN529 in the 1.4 mg/kg dose group was approximately 47.2 hours.

### Example 2

### IMGN529 Prophylaxis

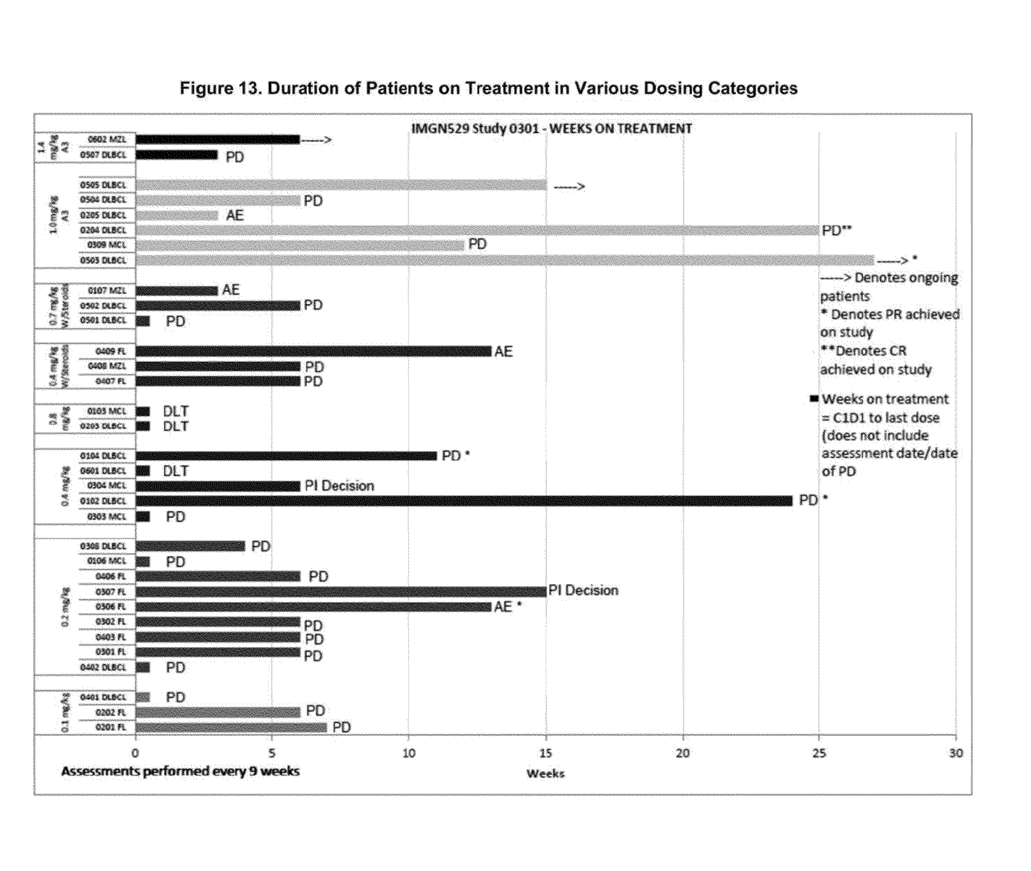

The protocol was amended to include peri-infusional corticosteroids. Re-escalation with corticosteroids was started at the 0.4 mg/kg IMGN529 dose level followed by 0.7 mg/kg IMGN529. No DLTs were observed at either dose, as shown in FIG. 11A. At the 1.0 mg/kg IMGN529 dose level one patient presented with Grade 3 febrile neutropenia on day 12 of the first cycle (C1D12), which constitutes a DLT, and two patients presented with Grade 4 neutropenia later in the cycle. Prophylactic use of peri-infusional corticosteroid helped to mitigate the occurrence of early onset neutropenia, and no febrile neutropenias have been reported since the protocol was amended to include peri-infusional corticosteroids and G-CSF. At the 1.0 mg/kg CD37-3-SMCC-DM1 dose level, two patients with DLBCL who were heavily pretreated and who relapsed following autologous transplant achieved an objective response. One patient achieved a partial response and one patient achieved a complete response. Furthermore, the addition of the corticosteroids to the dosing protocol allowed a higher IMGN529 dose level to be administered. Patients stayed on the study longer, less neutropenia was observed between day 1 and day 5, and more clinical benefits were observed, as shown in FIG. 13.

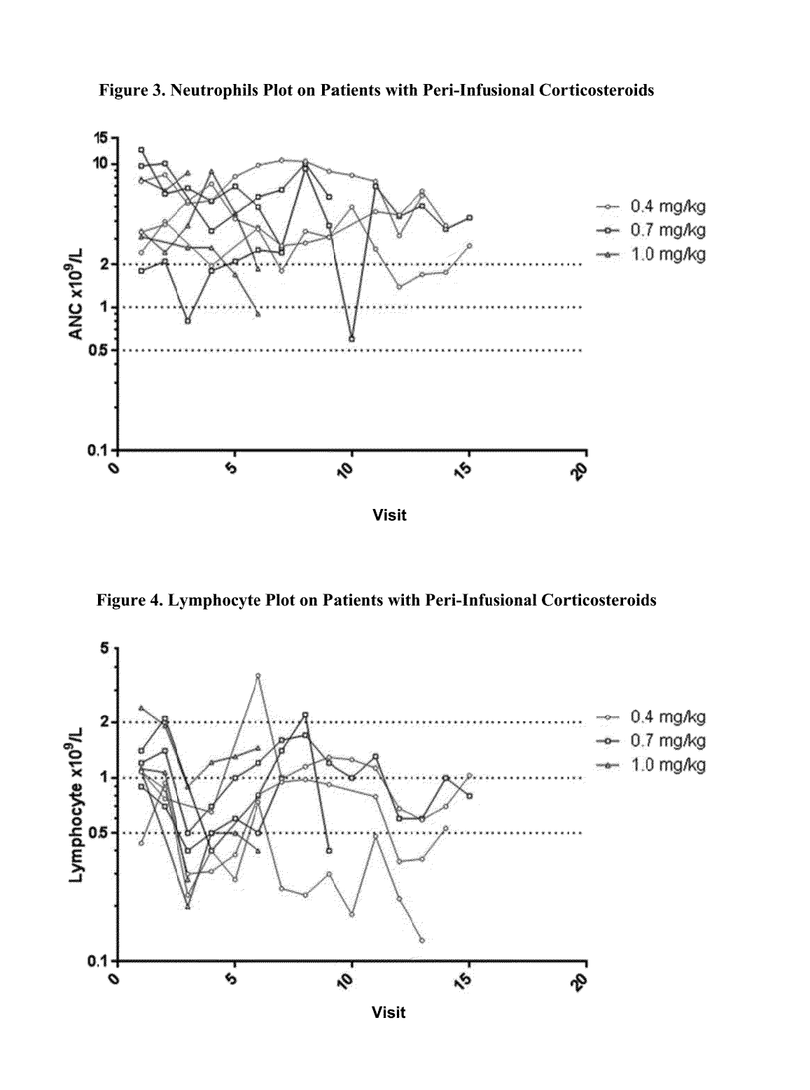

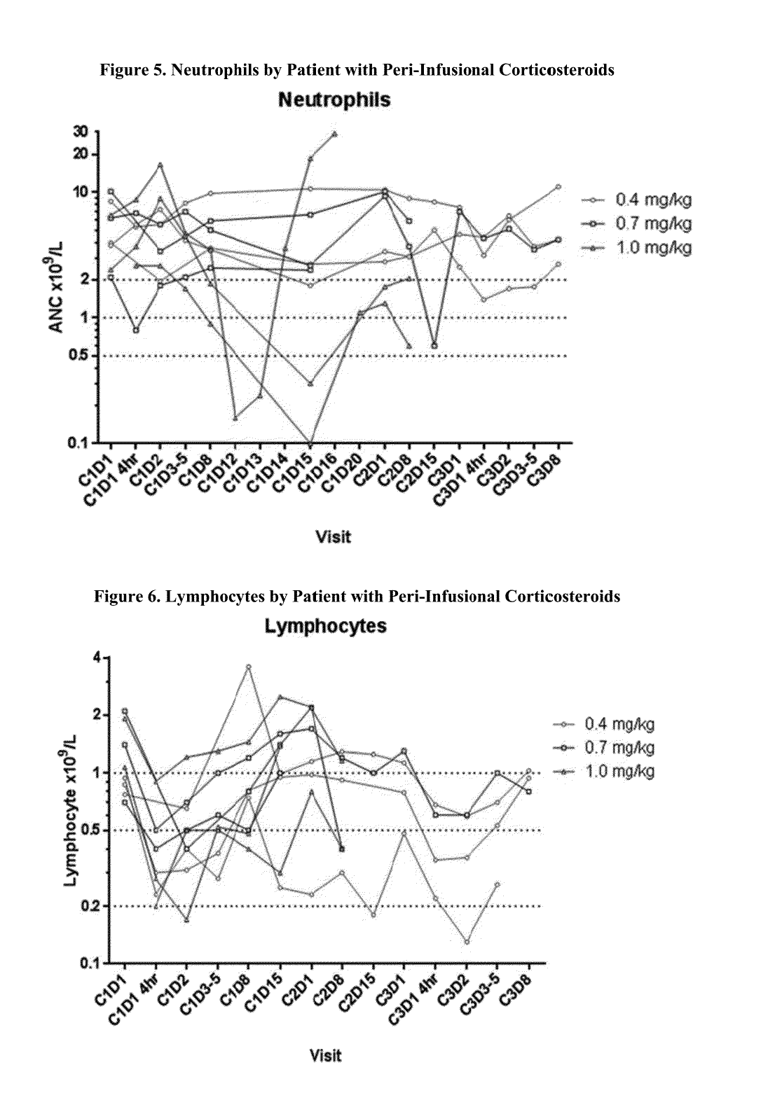

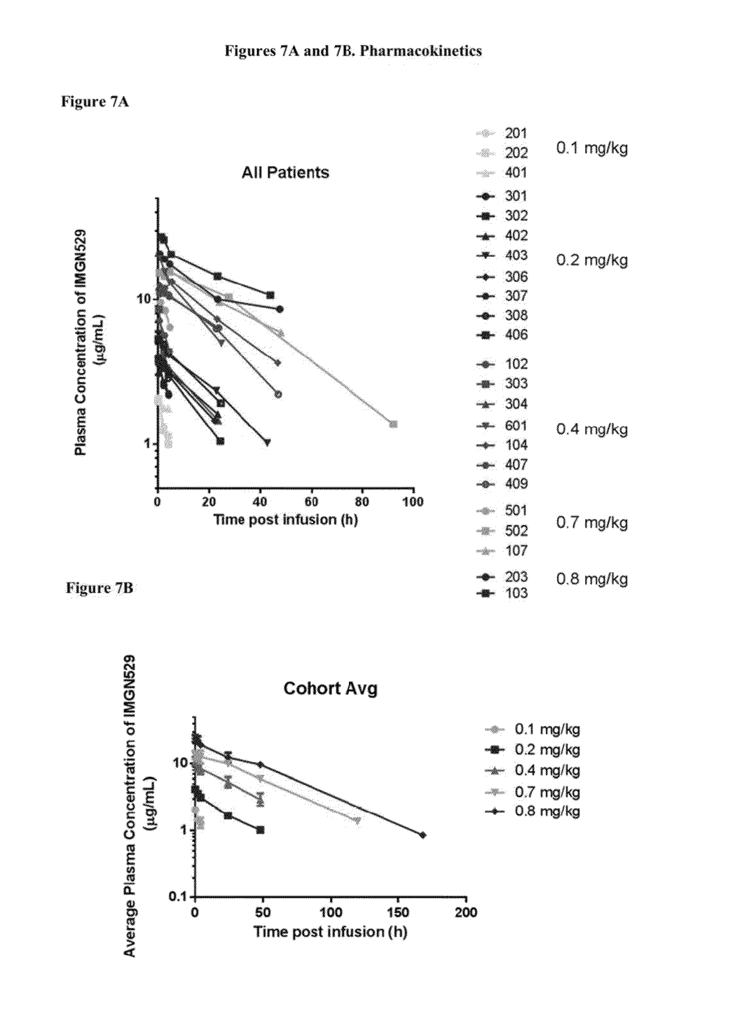

Absolute neutrophil counts (ANC) and lymphocyte levels in patients that received peri-infusional corticosteroids are shown in FIGS. 3 and 4, respectively. ANC and lymphocyte levels by Cycle and Day are shown in FIGS. 5 and 6, respectively. Drug exposure was measured in all patients and found to generally increase with dose (FIGS. 7A-B). Administration of peri-infusional corticosteroids does not appear to have an impact on pharmacokinetics.

### Example 3

### IMGN529 Growth Factor-Based Prophylaxis

In order to decrease the likelihood of neutropenia, any of the following growth factor-based prophylaxis protocols can be used.

(1) Patients receive G-CSF after administration of IMGN529.
G-CSF can be administered, for example, at a dose of about 5 μg/kg/day (e.g., 24 to 72 hours after administration of IMGN529).
G-CSF can be administered, for example, at a dose of about 480 μg/day (e.g., about 1 to 14 or about 5 to 14 or about 8 to 14 days after administration of IMGN529).
G-CSF can be administered, for example, at a dose of about 300 μg/day (e.g., about 1 to 14 or about 5 to 14 or about 8 to 14 days after administration of IMGN529).

(2) Patients receive GM-CSF after administration of IMGN529.
GM-CSF can be administered, for example, at a dose of about 250 μg/m²/day (e.g., 24 to 72 hours after administration of IMGN529).
GM-CSF can be administered, for example, at a dose of about 480 μg/day (e.g., about 1 to 14 or about 5 to 14 or about 8 to 14 days after administration of IMGN529).
GM-CSF can be administered, for example, at a dose of about 300 μg/day (e.g., about 1 to 14 or about 5 to 14 or about 8 to 14 days after administration of IMGN529).

(3) Patients receive pegfilgrastim after administration of IMGN529.
Pegfilgrastim can be administered, for example, at a dose of about 6 mg (e.g., about 24 hours after administration of IMGN529).
Pegfilgrastim can be administered, for example, at a dose of about 100 μg/kg (e.g., about 24 hours after administration of IMGN529).

The G-CSF, GM-CSF, or pegfilgrastim is administered subcutaneously.

The addition of growth factor G-CSF to the dosing protocol mitigated febrile neutropenia. Administration of G-CSF also allowed for a higher IMGN529 dose to be administered. Therefore, patients were able to receive treatment for longer periods, and more clinical benefits were achieved.

### Example 4

### In Vitro B-Cell Depletion and In Vitro Cytokine Release

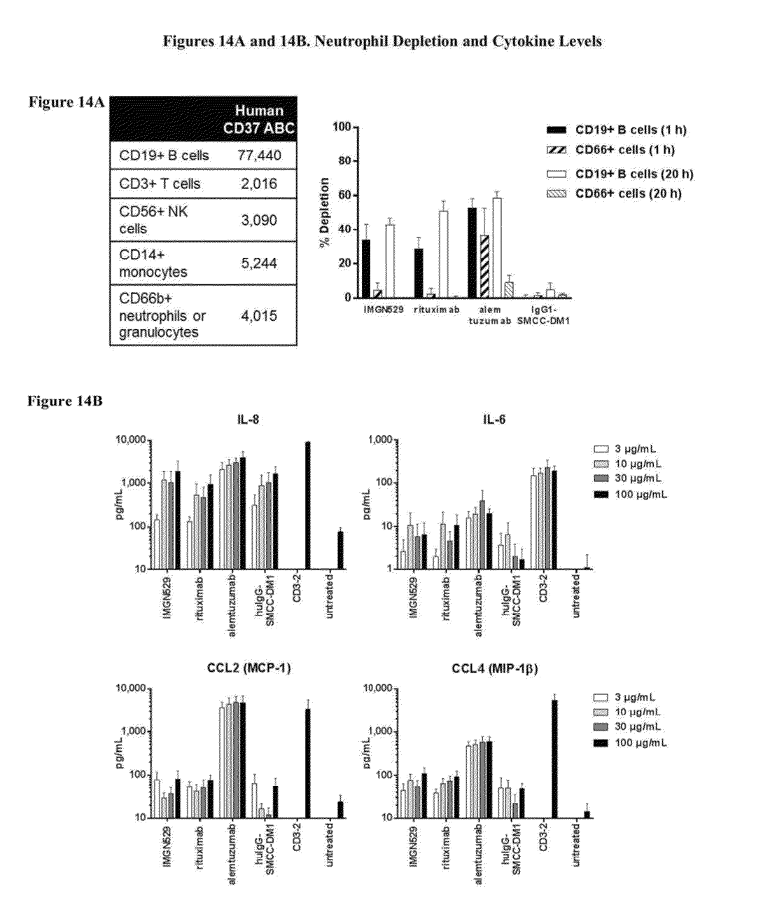

Effects of anti-human CD37 antibodies and immunoconjugates can be assessed by in vitro assays using human blood cells, using methods such as those provided in Deckert et al., 2013, Blood, 122(20):3500-3510. CD37 expression in peripheral blood cells was evaluated by quantitative flow cytometry using the commercially available QuantiBRITE system from BD Biosciences and the huCD37-3 antibody labeled with PE to estimate antigen density based on the number of antibodies bound to the cells (ABC). Fresh blood cells from four independent healthy donors were stained with approximately 10 μg/mL of huCD37-3-PE followed by red blood cell (RBC) lysis and analyzed in conjunction with QuantiBRITE PE beads according to the manufacturer's instructions. The average ABC values for the indicated blood cell populations were calculated and are listed in FIG. 14A. CD37 expression levels correspond to approximately 77,000 ABC on human B cells. CD37 was expressed at much lower levels of approximately 2,000 to 5,000 ABC in T cells, NK cells, monocytes, and granulocytes or neutrophils. This result demonstrates that high CD37 expression is mainly restricted to B-cells in peripheral blood samples with only minor expression on peripheral T cells, NK cells, monocytes, and granulocytes or neutrophils.

The ability of humanized antibodies to deplete B-cells was measured using in vitro assays with whole blood samples according to published studies (Vugmeyster et al., 2003, Cytometry A., 52(2):101-9 and Vugmeyster et al., 2004, Int Immunopharmacol., 4(8):1117-24). To identify populations of blood cells, all samples were incubated with 10 μg/mL of the treatments indicated in the bar graph in FIG. 14A for either 1 hour or 20 hours and then stained with fluorescently labeled anti-CD19-FITC for B-cells and anti-CD66-APC for granulocytes or neutrophils. Red blood cells were lysed and CountBright Absolute Counting Beads (Invitrogen) were added to each sample to allow standardization of cell counts. For three donors tested, treatment of blood samples with IMGN529 resulted in significant B-cell depletion with no apparent neutrophil depletion detected, similar to observations after rituximab (an anti-CD20 antibody) treatment (see FIG. 14A). In contrast, alemtuzumab (an anti-CD52 antibody) treatment in vitro resulted in both B-cell and neutrophil depletion. This is consistent with the high level of CD37 expression on target B cells and the relatively low CD37 expression level on other blood cells.

Cytokine levels were determined in culture supernatants of normal human blood cells following overnight treatment with 3-100 μg/mL of IMGN529, rituximab, alemtuzumab, a non-specific huIgG-SMCC-DM1 control conjugate, or an anti-CD3 antibody (CD3-2) using the cytometric bead array (CBA) method and commercially available BD FlexSet reagents according to the manufacturer's instructions. Analysis of cytokine release by peripheral blood cells from six normal human donors incubated with IMGN529 in vitro revealed increased levels of IL-8, CCL2 (MCP-1) and CCL4 (MIP-1β), but not IL-6 or TNF, to a similar extent as rituximab but less pronounced than alemtuzumab (see FIG. 14B).

### Example 5

### In Vivo Lymphocyte and Neutrophil Studies

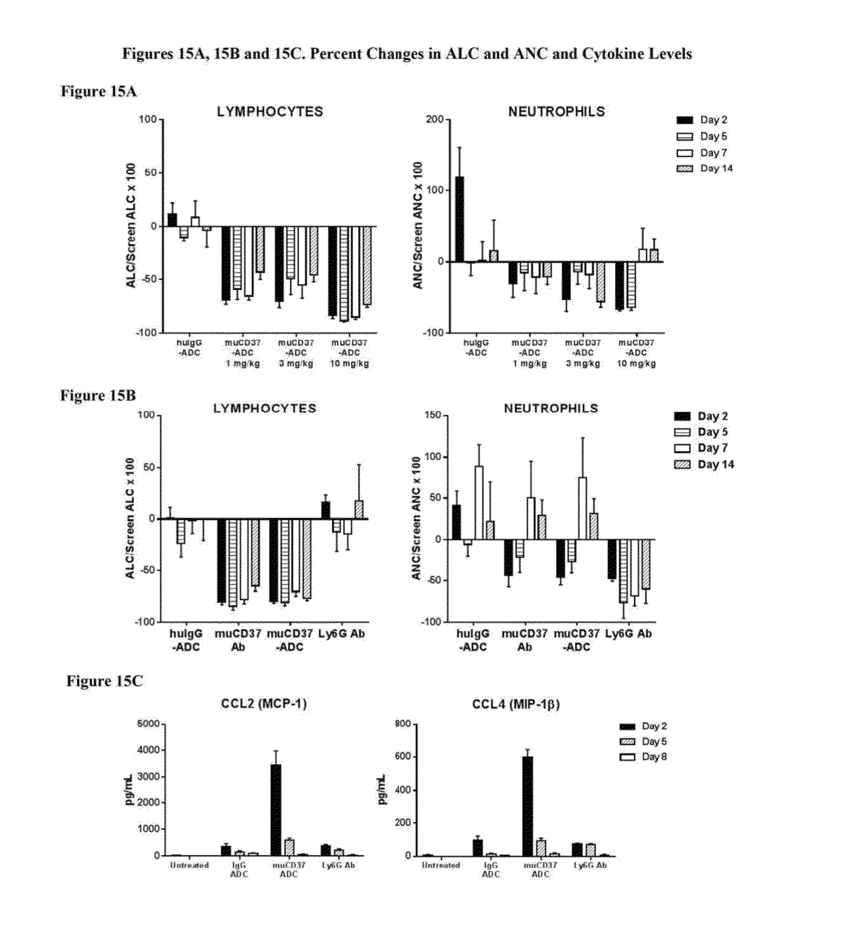

An anti-murine CD37 antibody (252-3) was used to characterize CD37 expression on murine blood cells and in in vivo studies in a murine model. Additional information regarding the murine antibody can be found in U.S. Patent Publication No. 2012/0276119 A1, which is incorporated by reference herein in its entirety. Similar to the expression profile of CD37 in human peripheral blood cells, CD37 expression on murine peripheral blood cells was highest in B cells, with much lower expression on T cells and granulocytes or neutrophils. In vivo activity of the anti-muCD37 antibody and the corresponding anti-muCD37-SMCC-DM1 conjugate (muCD37-ADC) was evaluated to discern antibody and payload-mediated events in comparison to the classic cytotoxic cyclophosphamide (CPA) and previously described neutrophil depleting anti-Ly6G antibody. C57/B6 mice were randomized into treatment groups, dosed with the agents indicated in FIG. 15A on day 1 and then bled via retro-orbital sinus at various time points to assess complete cell counts (CBC) in whole blood samples using the VetScan HM5 analyzer. This allows assessment of counts for white blood cells, lymphocytes, neutrophils, monocytes and platelets. For some studies percent changes in absolute lymphocyte counts (ALC) after treatment were calculated relative to cell counts at screening (screen) prior to treatment as follows: ((ALC after treatment/screen ALC)−1)*100. Percent changes in absolute neutrophil counts (ANC) were calculated accordingly.

Treatment of C57/B6 mice with 1-10 mg/kg of anti-muCD37 ADC resulted in a significant decrease in ALC lasting greater than 7 days and a more transient decrease in ANC (see FIG. 15A). Treatment of C57/B6 mice with 10 mg/kg of anti-muCD37 antibody had a similar effect as treatment with 10 mg/kg of anti-muCD37 ADC (see FIG. 15B). A non-targeted control SMCC-DM1 ADC had no effect on ALC or ANC counts, showing that the decrease is a CD37-mediated effect. In comparison, treatment with anti-Ly6G antibody resulted in more sustained ANC decline than seen with anti-muCD37-ADC (see FIG. 15B). However, treatment with anti-muCD37 ADC had no impact on neutrophils in mouse spleen in comparison to a non-targeted control ADC or the anti-Ly6G antibody.

Murine cytokine and chemokine levels were evaluated after treatment with muCD37-ADC in comparison to a non-targeted control SMCC-DM1 ADC and anti-Ly6G antibody using the cytometric bead array (CBA) method and commercially available BD FlexSet reagents according to the manufacturer's instructions. Elevated levels of CCL2 (MCP-1) and CCL4 (MIP-1β) chemokines were detected in mouse plasma after anti-muCD37 ADC treatment (see FIG. 15C). Elevation of these chemokines can contribute to a redistribution of circulating neutrophils into peripheral tissues.

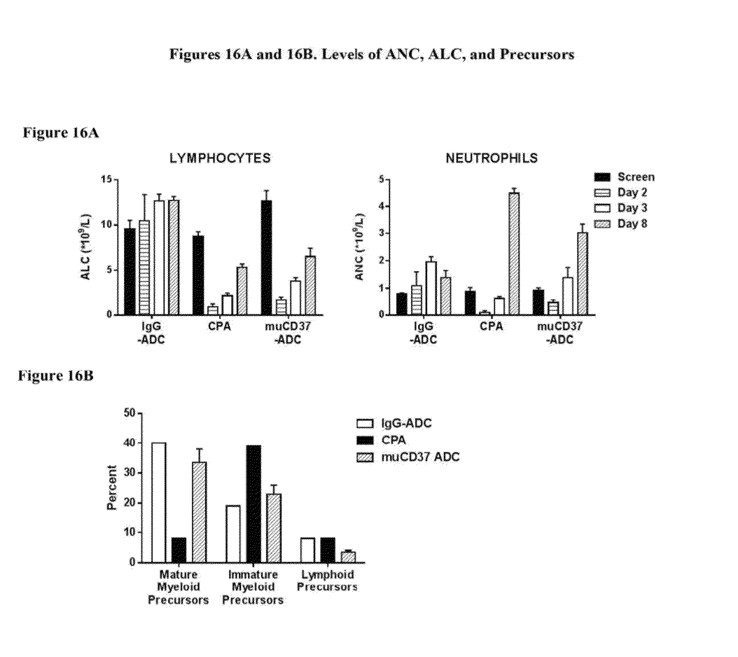

In contrast, treatment of C57/B6 mice with CPA resulted in an ALC decrease similar to the effect seen after treatment with muCD37-ADC, but CPA resulted in a more pronounced ANC decline (see FIG. 16A). Cellular content of bone marrow following treatment was evaluated by a pathologist via standard bone marrow smears and Giemsa staining to identify the percentage of various precursor cell populations. No impact on bone marrow lymphocyte, myeloid, or erythroid precursor cell counts was observed in response to the anti-muCD37 ADC, whereas CPA treatment caused reduced cellularity with a decrease in the percentage of mature myeloid precursors and neutrophils in bone marrow (see FIG. 16B).

Alternatively, the impact of anti-human CD37 antibodies and immunoconjugates can also be tested in murine models that have been engineered to express the human CD37 antigen. Such human CD37 (huCD37) expressing mice can be generated using standard knock in (KI) or transgenic (Tg) approaches. For example, to generate huCD37 KI mice, human CD37 cDNA can be inserted into the murine CD37 locus in the C57/B16 embryonic stem (ES) cells. The homozygous huCD37 KI mice will express human CD37 cDNA under the regulation of the endogenous murine CD37 promoter, thus the expression pattern of the huCD37 would mimic that of the endogenous muCD37. A different approach utilizes bacterial artificial chromosome (BAC) containing the human CD37 gene that can be randomly inserted into the mouse genome yielding huCD37-transgenic mice that express the human CD37 gene under the regulation of the human CD37 promoter.

It is to be appreciated that the Detailed Description section, and not the Summary and Abstract sections, is intended to be used to interpret the claims. The Summary and Abstract sections sets forth one or more, but not all, exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

1. A method for treating a human patient having cancer comprising administering to the patient a therapeutically effective dose of an immunoconjugate which binds to CD37 polypeptide, wherein the immunoconjugate is administered at a dose of from about 0.1 mg/kg to about 3.0 mg/kg, wherein the immunoconjugate comprises an antibody or antigen-binding fragment thereof comprising a variable heavy chain comprising the CDR1, CDR2, and CDR3 sequences set forth in SEQ ID NOs: 4-6, respectively and a variable light chain comprising the CDR1, CDR2, and CDR3 sequences set forth in SEQ ID NOs: 7-9, respectively.

2-3. (canceled)

4. The method of claim 1, wherein the antibody comprises the variable heavy chain sequence of SEQ ID NO:1.2 and the variable light chain sequence of SEQ ID NO: 15.

5. (canceled)

6. The method of claim 1, a wherein the immunoconjugate comprises a maytansinoid.

7. (canceled)

8. The method of claim 1, where the immunoconjugate comprises an SMCC linker.

9. (canceled)

10. The method of claim 1, wherein the immunoconjugate is IMGN529.

11-13. (canceled)

14. The method of claim 1, wherein the immunoconjugate is administered at a dose of from about 0.4 mg/kg to about 1.4 mg/kg.

15. (canceled)

16. The method of claim 1, wherein the immunoconjugate is administered at a dose of from about 1.4 mg/kg to about 2.0 mg/kg.

17. The method of claim 1, wherein the immunoconjugate is administered at a dose of from about 2.0 mg/kg to about 2.8 mg/kg.

18-26. (canceled)

27. The method of claim 1, wherein the immunoconjugate is administered once every three weeks.

28-29. (canceled)

30. The method of claim 1, further comprising administering a corticosteroid to the patient.

31. The method of claim 30, wherein the corticosteroid is administered prior to the administration of the immunoconjugate.

32. (canceled)

33. The method of claim 30, wherein the corticosteroid is administered peri-infusionally.

34. The method of claim 30, wherein the corticosteroid is administered during the administration of the immunoconjugate.

35-38. (canceled)

39. The method of claim 30, wherein the corticosteroid is administered after the administration of the immunoconjugate.

40. The method of claim 30, wherein the corticosteroid is selected from the group consisting of prednisone, prednisolone, methylprednisolone, beclamethasone, betamethasone, dexamethasone, fludrocortisone, hydrocortisone, and triamcinolone.

41-47. (canceled)

48. The method of claim 1, wherein the cancer is leukemia or lymphoma.

49. The method of claim 1, wherein the cancer is selected from the group consisting of B-cell lymphomas including NHL, precursor B-cell lymphoblastic leukemia/lymphoma and mature B-cell neoplasms, such as chronic lymphocytic leukemia (CLL)/small lymphocytic lymphoma (SLL), prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B-cell lymphoma (MALT type, nodal and splenic type), hairy cell leukemia, diffuse large B-cell lymphoma (DLBCL), Burkitt's lymphoma, plasmacytoma, plasma cell myeloma, post-transplant lymphoproliferative disorder, Waldenstrom's macroglobulinemia, and anaplastic large-cell lymphoma (ALCL).

50-62. (canceled)

63. A method of treating chronic lymphoid leukemia (CLL) comprising administering to a human patient in need thereof a therapeutically effective dose of an immunoconjugate which binds to CD37 polypeptide once every three weeks, wherein the immunoconjugate comprises an antibody or antigen-binding fragment thereof comprising a variable heavy chain comprising the CDR1, CDR2, and CDR3 sequences set forth in SEQ ID NOs: 4-6, respectively and a variable light chain comprising the CDR1, CDR2, and CDR3 sequences set forth in SEQ ID NOs: 7-9, respectively, and wherein the immunoconjugate is administered at a dose of from about 0.1 mg/kg to about 3.0 mg/kg.

64. (canceled)

65. The method of claim 63, wherein the immunoconjugate is administered at a dose of from about 1.4 to about 2.0 mg/kg.

66. The method of claim 63 further comprising administering to the human patient a peri-infusional corticosteroid.

67. A method for treating a human patient having cancer comprising administering to the patient a therapeutically effective dose of an immunoconjugate which binds to CD37 polypeptide and a growth factor, wherein the immunoconjugate is administered at a dose of from about 0.1 mg/kg to about 3.0 mg/kg, wherein the immunoconjugate comprises an antibody or antigen-binding fragment thereof comprising a variable heavy chain comprising the CDR1, CDR2, and CDR3 sequences set forth in SEQ ID NOs: 4-6, respectively and a variable light chain comprising the CDR1, CDR2, and CDR3 sequences set forth in SEQ ID NOs: 7-9, respectively.

68. The method of claim 67, wherein the growth factor is selected from the group consisting of granulocyte colony-stimulating factor (G-CSF), granulocyte-macrophage colony-stimulating factor (GM-CSF), macrophage colony-stimulating factor (M-CSF), filgrastim, and pegfilgrastim.

69. The method of claim 67, wherein the growth factor is administered at least once from day one to day twelve after administration of the immunoconjugate.

70. The method of claim 67, wherein the growth factor is administered at least one from day 15 to day 21 after administration of the immunoconjugate.
